(19) United States
(12) Patent Application Publication
(10) Pub. No.: US 2007/0224077 A1
(43) Pub. Date: Sep. 27, 2007

(54) Heat Processing Systems, Apparatuses, and Methods for Collection and Disposal of Infectious and Medical Waste

(75) Inventors: Donald G. Cox, Franktown, CO (US); Diane R. Gorder, Monument, CO (US)

Correspondence Address: JOHN S. PRATT, ESQ;KILPATRICK STOCKTON, LLP, 1100 PEACHTREE STREET, ATLANTA, GA, 30309, US

(73) Assignee: BioMedical Technology Solutions, Inc., Englewood, CO (US)

(21) Appl. No.: 11/689,638

(22) Filed: Mar. 22, 2007

Related U.S. Application Data
(60) Provisional application No. 60/785,512, filed on Mar. 23, 2006.
(60) Provisional application No. 60/785,548, filed on Mar. 23, 2006.

(51) Int. Cl. A61L 2/04

(52) U.S. Cl. 422/1; 422/307

(57) ABSTRACT

Various embodiments of systems and methods for collection and disposal of infectious and medical waste are disclosed. An embodiment includes a system with a body having a chamber that receives a container of medical waste. The chamber may include a canister that has limited access to the interior of the canister for safe collection of sharps material. The chamber may have at least one plate heater coupled thereto for providing heat to the chamber and a plurality of fins on the chamber to assist in cooling the chamber.

This application claims priority to U.S. Application Ser. No. 60/785,512 filed on Mar. 23, 2006, and U.S. Application Ser. No. 60/785,548 filed on Mar. 23, 2006, the entire contents of each of which are hereby incorporated by this reference.

### FIELD OF THE INVENTION

This invention relates to devices, systems, and methods for collection and disposal of infectious and medical waste, and more particularly to devices for safe and tamper-resistant collection and disposal of medical waste and rendering infectious and medical waste safe and sterile using heat processing systems and methods.

### BACKGROUND OF THE INVENTION

The safe handling and disposal of regulated medical waste from various medical and health care facilities is a well-known problem. Numerous environmental regulations prevent the use of conventional methods of waste disposal, while many on-site methods to render the infectious and medical waste safe have not proven to be practical or cost-efficient.

Of particular concern is the safe collecting and processing of contaminated needles, scalpels, and sharp metal or glass objects that have come into contact with the human body or bodily fluids. These items often include thermoplastic materials such as those found in syringes and tubing, vials of glass, and other objects that have contacted bodily fluids. The problems associated with used thermoplastic hypodermic needles and syringes are well known. Collection and disposal of medical waste must be carefully controlled to prevent needlestick injury exposure or reuse that could lead to serious illness or even death. Past disposal techniques involve the requirement of medical facilities to cut the needle from the syringe body immediately after injection. This procedure, however, was discovered to spread disease through airborne aerosols caused by the mechanical sheering action. The contaminated needle tip and syringe would then still need to be handled and disposed of as a regulated waste item. More recent developments have led to depositing the syringe and needle into a “sharps” container. The sharps container would then be delivered to an authorized facility in a costly “tracking,” treatment, and disposal process.

Existing methods and systems, such as incineration, autoclaving, chemical treatment, electronic beam radiation, gamma rays, microwave energy, use of a low voltage electric current to destroy a needle at the point of use, encasing needles in resins or gels, and the like, have numerous shortcomings. Those shortcomings include inefficiency, high cost, high possibility for human error, inability to handle both sharps and red bag medical waste, and creation of infectious and hazardous fumes when the medical waste is treated.

For example, devices for destroying a needle with a low voltage electric current do not have the capability to render safe other commonly used materials, such as scalpels, glass, and leftover syringe parts. As another example, techniques for using resins or gels to encapsulate needles typically involve melting a thermoplastic bag with waste at an autoclave temperature. However, the product of such a system remains a hazardous material for handling purposes, as the treated waste is recognizable and can be unsterile. Moreover, autoclave sterilization depends on “wet heat” destroying microbial life by having the heat contact the life forms for a defined period, but this process is not efficient when the waste is shielded by plastic bags or immersed in a melted plastic. As another example, many systems discharge infectious and hazardous fumes when the waste material is heated. Finally, many existing systems are capable of handling syringes, but not capable of handling soft waste, such as gauze, tape, and fabrics for sterilization purposes in a single on-site system.

The methods and systems disclosed in U.S. Pat. No. 5,972,291, which is hereby incorporated by reference in its entirety, for collecting and heat processing infectious and medical waste addressed many of the concerns associated with preexisting devices, methods and systems for collection and disposal of infectious and medical waste. However, there remains a need for improved devices, methods and systems for collecting and disposing of infectious and medical waste of all types, including, but not limited to, even more efficient processing, improved heat transfer between the heat chamber and the container with waste material that is being treated, improved cooling of the heat chamber and heat processing system, and enhanced process data and quality control monitoring systems to support compliance and system reliability and performance.

### SUMMARY OF THE INVENTION

The present invention provides devices, systems, and methods for safe and effective collection and disposal of infectious and medical waste, as well as disposal of such waste using heat treatment. In one embodiment, a system for thermal processing of infectious and medical waste comprises a body having a chamber to receive a container of medical waste, at least one plate heater coupled to an exterior surface of the chamber for providing heat to the container, and a plurality of fins formed on an exterior surface of at least one side of the chamber for cooling the chamber. The system may also include a filter within the body and having an inlet coupled to the chamber. Two plate heaters may be provided, each plate heater coupled to an exterior surface of opposite sides of the chamber. The system may further include a plurality of fins formed in the exterior surfaces of the opposite sides of the chamber adjacent to where the two plate heaters are coupled and/or a plurality of fins formed in an exterior surface on a bottom of the chamber. The two sides and the bottom on which the plurality of fins are formed on the chamber may be extruded.

In certain embodiments, the chamber is shaped such that a plurality of interior surfaces of the chamber contact a plurality of exterior surfaces of a container of medical waste when the container is received within the chamber. A system may further comprise the container for receiving medical waste, and the container may comprise a canister with a plug sealing a top end of the canister. In one embodiment, the medical waste canister may be fabricated from a thermoplastic material such that exposure to dry heat will result in the canister melting to encapsulate its contents. In such embodiments, a sleeve may be included to hold the container, and both the sleeve and container may be receivable within the chamber. The sleeve may be reusable and coated with a non-stick material on its interior and will fully contain the melted waste canister after processing. In another embodiment, a canister, rim, and plug are made of materials that withstand the dry heat cycle applied using such a system, such that they do not melt, while a chute and member in the canister are made of a plastic material that melts and encapsulates the waste material, as further described below.

In certain embodiments, a heat treatment system may include an exhaust tube extending from the chamber for exhaust resulting from heating the waste. A second tube may be coupled to the exhaust tube, the second tube including a spiral portion that ends at a T-valve, wherein the exhaust from the chamber is separated into gas and condensed water vapor in the second tube. A third tube may be coupled to one outlet of the T-valve and a fourth tube coupled to another outlet of the T-valve, the third tube for passing gas to a filter and the fourth tube for passing liquid to a collection receptacle.

In one embodiment, a system may further comprise a processor coupled to an external link capable of contact with a remote database and memory for storing data captured during a treatment cycle. The memory may include three data storage buffers, one for short term data, one for intermediate term data, and one for long term data.

In another embodiment, a method for heat-processing infectious and medical waste comprises heating a container of waste in a chamber to render the waste biologically safe using at least one plate heater coupled to an exterior surface of a side of the chamber and cooling the chamber using a plurality of fins positioned on an exterior surface of at least one side of the chamber and a plurality of fins positioned an exterior surface of a bottom of the chamber. In one embodiment, a method may further comprise providing the chamber to receive the container of waste, the chamber being shaped complementary to the shape of the container so that a plurality of exterior surfaces of the container contact a plurality of interior surfaces of the chamber when the container is received within the chamber. Two plate heaters may be used to heat the container of waste, the plate heaters coupled to exterior surfaces on each of two opposite sides of the chamber.

In certain embodiments, a plurality of fins positioned on an exterior surface of at least one side of the chamber comprises a plurality of fins positioned on exterior surfaces on each of two opposite sides of the chamber and an exterior surface of a bottom of the chamber. Exhaust resulting from heating the waste from the chamber may be separated into gas and condensed water vapor. In some embodiments, a method may include directing the gas through a tube to a filter and directing the condensed water vapor into a collection receptacle. In another embodiment, a method may include storing data related to a treatment cycle during which a container of waste is heated and cooled and transmitting at least a portion of the stored data to a remote database.

Certain embodiments of this invention may be used to collect waste that is to be disposed of or sterilized using a heat treatment process. In one embodiment, an apparatus for collection of infectious and medical waste comprises a canister comprising a top end with an opening for receiving waste, a rim configured to fit over an edge of and engage the top end of the canister, and a plug configured to engage the rim such that the plug seals the opening of the canister. The canister may include a plurality of slots spaced around its periphery, and the rim may include a plurality of projections that are received within the plurality of slots of the canister when the rim is engaged with the canister.

In certain embodiments, the rim includes a plurality of slots spaced around a periphery of an inner portion of the rim, and the plug has a plurality of hooks that are received within the plurality of slots of the rim when the plug is engaged with the rim. The plug also may include a plurality of tabs spaced around a periphery of the plug. These tabs backfill apertures may be found just above the projections of the rim when the rim, canister, and plug are engaged to ensure that engagement of the rim and canister is secure.

In another embodiment, an apparatus for collection of infectious and medical waste comprises a canister comprising a top end with an opening for receiving waste; a rim configured to fit over an edge of and engage the top end of the canister; a chute configured to engage an inner portion of the rim, the chute extending into an interior of the canister when engaged with the rim on the canister; a member movably mounted within the chute and configured to limit access to a portion of the interior of the canister beneath the member; and a plug configured to engage the rim such that the plug seals the opening of the canister. This embodiment is particularly useful in the handling of sharps material. In one embodiment, the chute and the member are made of a transparent or partially transparent thermoplastic material.

In certain embodiments, the chute includes a flange that sits upon an inner portion of the rim when the chute and the rim are engaged, and the flange may have a plurality of notches spaced around the flange and positioned to avoid interfering with engagement of the rim and the plug. In a preferred embodiment for use in a heat treatment system, the chute and member are made of a plastic material that melts and encapsulates the waste material, while the canister, rim, and plug are made of materials that withstand the dry heat cycle applied using such a system. The member may be mounted on posts of the chute and include a plurality of fins defining areas that receive the waste. Each fin is configured to rotate and dump waste into the portion of the interior of the canister beneath the member when waste is placed into one of the areas between the fins.

In certain embodiments, the canister is made using a thermoplastic material with a melting point at or below 340 degrees Fahrenheit. In such embodiments, the rim may be integrated into the outer body of the canister, including (as in other embodiments) mechanisms for attaching the chute and member. In such embodiments, for use in a heat treatment system, a plug is not used, and the canister/rim, chute, and member are all made of a plastic material that melts and encapsulates the waste material within the canister. When a canister is used in this manner, it may be placed within a reusable sleeve within the chamber of a heating system, as mentioned above.

In yet another embodiment, an apparatus for collection of infectious and medical waste comprises a canister comprising a top end with an opening for receiving waste; a rim configured to fit over an edge of and engage the top end of the canister; a chute configured to engage an inner portion of the rim, the chute extending into an interior of the canister when engaged with the rim atop the canister; and a member movably mounted within the chute and configured to limit access to a portion of the interior of the canister beneath the member.

Other embodiments are described and will become apparent from the detailed description below.

### BRIEF DESCRIPTION OF THE DRAWINGS

FIGS. 1A and 1B are perspective views of an embodiment of a heat processing system according to the present invention.

FIG. 2A is a top view of the heat processing system of FIG. 1.

FIG. 2B is schematic illustration of a cross-sectional side view of the heat processing system of FIG. 1, taken along line B-B shown in FIG. 2A, with a container of waste positioned within the heat chamber of the system.

FIG. 3 is a perspective view of the heat chamber of the system of FIG. 1.

FIG. 4 is a side view of the heat chamber of the system of FIG. 1.

FIG. 5 is an end view of the heat chamber of the system of FIG. 1.

FIG. 6 is a perspective view of the heat chamber of the system of FIG. 1, with a sealed container of waste within the heat chamber.

FIG. 7 is a perspective view of a portion of the system of FIG. 1 showing an exemplary heat chamber exhaust flow through the system.

FIGS. 8 and 9 are perspective views of portions of the system of FIG. 1 showing an exemplary ventilation air flow through the system.

FIGS. 10-12 are perspective views of portions of the system of FIG. 1 showing an exemplary cooling air flow through the system.

FIG. 13 shows a block diagram of an exemplary embodiment of data storage, processor, and external link components of a system according to the present invention.

FIG. 14 is a perspective view of an embodiment of a canister according to the present invention.

FIG. 15 is a perspective view of an embodiment of a rim according to this invention.

FIG. 16 is another perspective view of the rim of FIG. 15.

FIG. 17 is a perspective view of the canister of FIG. 14 and rim of FIG. 15 assembled.

FIG. 18 is a perspective view of an embodiment of a plug according to this invention.

FIG. 19 is a perspective view of the canister of FIG. 14, the rim of FIG. 15, and the plug of FIG. 18 assembled.

FIG. 20 is a perspective view of the rim of FIG. 15 assembled with the plug of FIG. 18 from underneath the assembly.

FIG. 21 is a perspective, exploded view of an embodiment of a chute and spinner according to this invention, along with the rim of FIG. 15 and the plug of FIG. 18.

FIG. 22 is a perspective view of the chute and spinner of FIG. 21 fully assembled.

FIG. 23 is an end view of the spinner of FIG. 21.

FIGS. 24A and 24B are perspective and side views, respectively, of the chute and spinner of FIG. 21 assembled with the rim of FIG. 15 and the plug of FIG. 18.

FIG. 25 is a perspective view of the chute of FIG. 21 and rim of FIG. 15 assembled with the canister of FIG. 14.

FIG. 26 is a perspective view of the chute and spinner of FIG. 21 and the rim of FIG. 15 assembled with the canister of FIG. 14.

FIG. 27 is a perspective view of another embodiment of a chute according to the present invention.

FIG. 28 is a perspective view of an embodiment of an outer container according to the invention, with the canister of FIG. 14 positioned within the outer container and the rim of FIG. 15 and the chute and spinner of FIG. 21 assembled with the canister.

FIG. 29 is a perspective view of the outer container of FIG. 28, with its top open.

FIG. 30 is a front view of the outer container of FIG. 28, with its top open.

FIGS. 31A-31C are end, front, and end views of the outer container of FIG. 28, with its top closed.

FIG. 32 is a perspective view of the outer container of FIG. 28, with its top closed.

FIG. 33 is a perspective view of the outer container of FIG. 28, with its top closed, from underneath the outer container.

FIGS. 34A-34B show end and perspective views, respectively, of another embodiment of a spinner according to this invention.

FIGS. 35A-35B show end and perspective views, respectively, of an embodiment of an insert that fits within ends of the spinner of FIGS. 34A-34B.

FIG. 35C shows an exploded perspective view of the spinner of FIGS. 34A-34B and the insert of FIGS. 35A-35B.

FIG. 36 shows a perspective exploded view another embodiment of a canister and a sleeve that may be positioned within the chamber (shown in FIGS. 3-6) of a heat processing system according to the present invention.

FIG. 37 shows a perspective view of another embodiment of a spinner according to this invention.

### DETAILED DESCRIPTION OF THE INVENTION

The present invention provides systems, devices, and methods for collection and disposal of infectious and medical waste using heat treatment. Certain exemplary embodiments of this invention comprise medical waste treatment systems capable of processing infectious medical waste, except human and animal body parts, radioactive waste, and chemotherapeutic waste (depending on state regulations). In general, medical waste is comprised of different types of components such as plastic, cotton, aluminum, glass, etc. Generally, “sharps” material refers to needles, syringes, IV tubing, and the like, while soft or “red bag” waste refers to gauze, cotton balls, tubing, gowns, etc., which may be contaminated with potentially infectious fluids such as blood and the like.

Certain embodiments of this invention are useful on-site in a variety of settings where medical waste may be generated, including, but not limited to outpatient medical and dental clinics, long term care facilities, home health care, public health care facilities, veterinary facilities, military facilities, and the like. Certain embodiments of the present invention provide a single on-site system to handle sterilization and/or destruction of all types of regulated medical waste.

For example, in the United States alone, there are well over half a million private, outpatient medical and dental offices. Routine patient treatments include a wide variety of invasive and non-invasive procedures that generate potentially infectious biomedical wastes including disposable examination materials, injection/immunization sharps, specimen collection and testing tools, disposable elective surgery materials, disposable dental cleaning materials, and the like. Without proper and efficient processing, accumulation, storage, and carting of these wastes can result in unnecessary exposure risk to both patients and staff.

As another example, U.S. population statistics clearly indicate the growing need for long term healthcare. Currently there are more than 40,000 skilled nursing care facilities in the United States alone, including nursing homes, assisted living facilities and hospices. It is estimated that treatment of each patient in a long term care facility will produce approximately 8 ounces of biomedical waste, per day. With the increased need for long term care, there is a more pressing need for safe and efficient processing of these potentially infectious wastes.

As another example, the need for home health care results from a variety of conditions and extends into a wide range of population demographics. For example, insulin dependent diabetics will self-administer at least 3 billion injections per year in the U.S. alone, and, worldwide, discarded needles and syringes from some 8 billion self-administered injections are improperly discarded posing risk of injury and infection. The industry for in-home care, for example for the elderly, chronic disease, acute conditions, rehabilitation, and end-of-life support, has grown at the rate of 20% per year for more than 10 years. Although many medical advances in the miniaturization and portability of medical equipment have been made, innovations in the management of potentially infectious medical waste produced from home health care have not been adequately addressed until now.

As yet another example, there are virtually hundreds of thousands of public health care settings where biomedical waste is generated in the form of both sharps and red bag biomedical waste, including public health clinics, urgent care clinics, school health clinics, first aid stations, ambulances, air evacuation vehicles, public venues (stadiums, airports, cruise lines, etc.), pharmacies, and the like. Additionally, restrooms located in public settings often serve as a makeshift facility for self-treatment, producing potentially infectious waste that should be properly processed for safe disposal.

As yet another example, there are approximately 55,000 veterinarians in private practice in the United States alone. An additional 8,000 treat animals as part of their role in academic and government positions. In veterinary medicine, potentially infectious wastes are produced as a result of routine examination, immunizations and treatment. In light of growing concern regarding the danger of zoonotic disease, proper handling and disposal of such wastes are necessary for the safety of both veterinary staff and waste management personnel, including at settings such as veterinary clinics, hospitals and emergent care centers; schools of veterinary medicine, mobile veterinary practices, veterinary/animal research centers, animal rescue organizations, mobile veterinary medical assistance teams, and the like.

Further examples include military medicine and health care for developing countries. There are approximately 1.5 million active duty military personnel in the United States armed forces and another 1.2 million on the reserve rolls. Healthcare in support of military missions is frequently dispensed by means of mobile services (air evacuation, naval ships, ambulance) and field care (temporary treatment facilities). Additionally, over 16 billion injections are administered each year worldwide. In certain under-developed countries, as many as 70% of these injections may be delivered in unsterile conditions. In India alone dozens of new Hepatitis and thousands of new HIV/AIDS cases are reported as a result of unsafe injection practices, primarily due to re-use of contaminated needles/syringes.

In use, certain embodiments of a canister with medical waste in it are placed into a heat chamber of certain embodiments of a heat processing system according to this invention. In preferred embodiments, the shape of the canister or container (or sleeve, if the canister is to be placed within a sleeve, as described below) and the heat chamber are complementary, allowing for direct heat conduction between the sides of the heat chamber and the sides of the container (or sleeve). In certain embodiments, a chamber that holds a container of medical waste is heated to a temperature of between about 300 degrees and about 425 degrees Fahrenheit so that plastic portions of the waste begin to at least warp. Preferably, the chamber is heated to a temperature of between about 325 and about 425 degrees Fahrenheit, and more preferably between about 350 and about 400 degrees, to melt all the plastic portions of the waste. If heated to melt, upon hardening, the melted thermoplastic material becomes a biologically sterile and unitary mass in which the sharp edges and points of syringes, tubes, and needles are at least partially encapsulated within the resin. The sharps material is rendered unrecognizable and unreusable and is sterile due to the heating and hardening process, thereby allowing it to be disposed of as ordinary garbage.

In one embodiment, while the sharps waste is partially encapsulated inside the canister, the entire canister holding the treated waste, with for example a rim and plug (described below) sealing the canister, is disposed of as ordinary solid waste. Exposure of red bag waste to this amount of heat renders the waste sterile, and therefore allows it to be disposed of with ordinary garbage as well. In other words, the sealed canister survives the heat treatment process. It should be understood, however, that the present invention is not limited to collection devices for use only with heat systems, but rather certain embodiments of this invention may be used for stand-alone collection and containment of medical waste, which may then be sterilized or permanently disposed in a number of other suitable manners.

In another embodiment, the medical waste canister is fabricated from a thermoplastic material such that upon application of dry heat during the processing of the waste, the canister itself (not just a chute and/or spinner at the top) is intended to melt and encapsulate its contents. In these embodiments, the rim may be integrated into the outer body, including as in other embodiments means for attaching the chute and member. In these alternative embodiments for use in a heat treatment system, the plug may be obviated, and the canister/rim, the chute and member are all made of a plastic material that melts and encapsulates the treated waste material. In such embodiments, a sleeve into which the medical waste canister can be placed is used, rather than placing the canister directly into the heat chamber of the treatment system. This sleeve may be fabricated from a material suitable to repeated usage and removal of processed, melted canisters. In one embodiment, the sleeve is constructed of aluminum and the interior coated with a non-stick material. The sleeve may be dimensioned such that its sides contact the sides of the heat chamber when inserted to allow direct conduction of heat from the heat chamber to the sleeve. To process a canister of such an embodiment, it may be inserted into the sleeve, and the sleeve/canister combination inserted into the heat chamber. After processing is complete, the sleeve may be removed from the heat chamber, and the processed contents discharged therefrom.

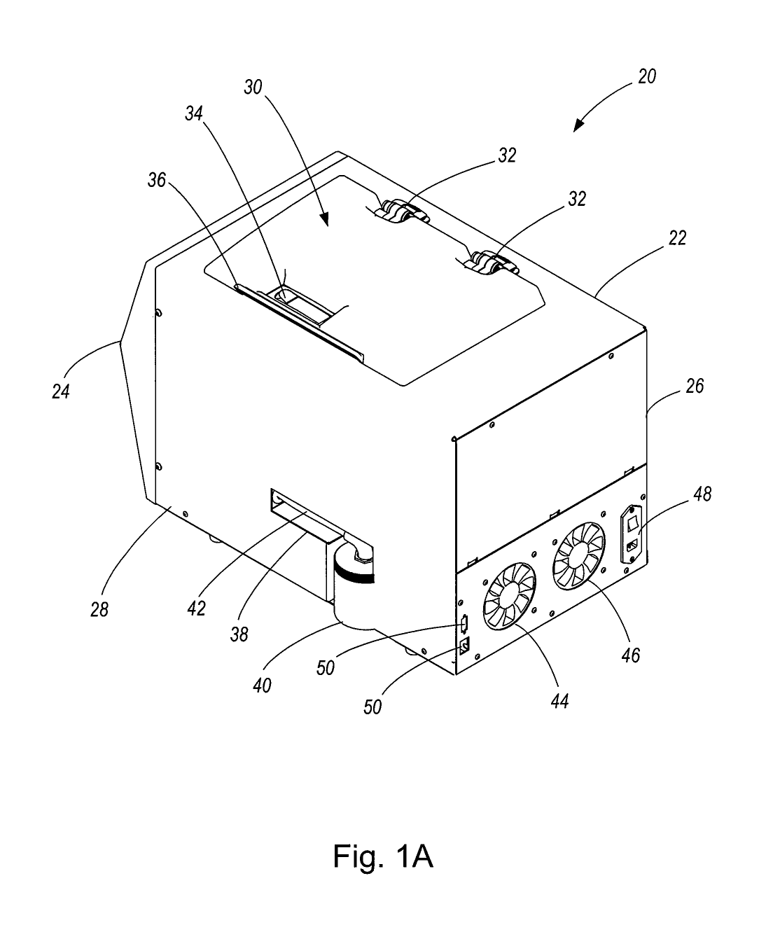

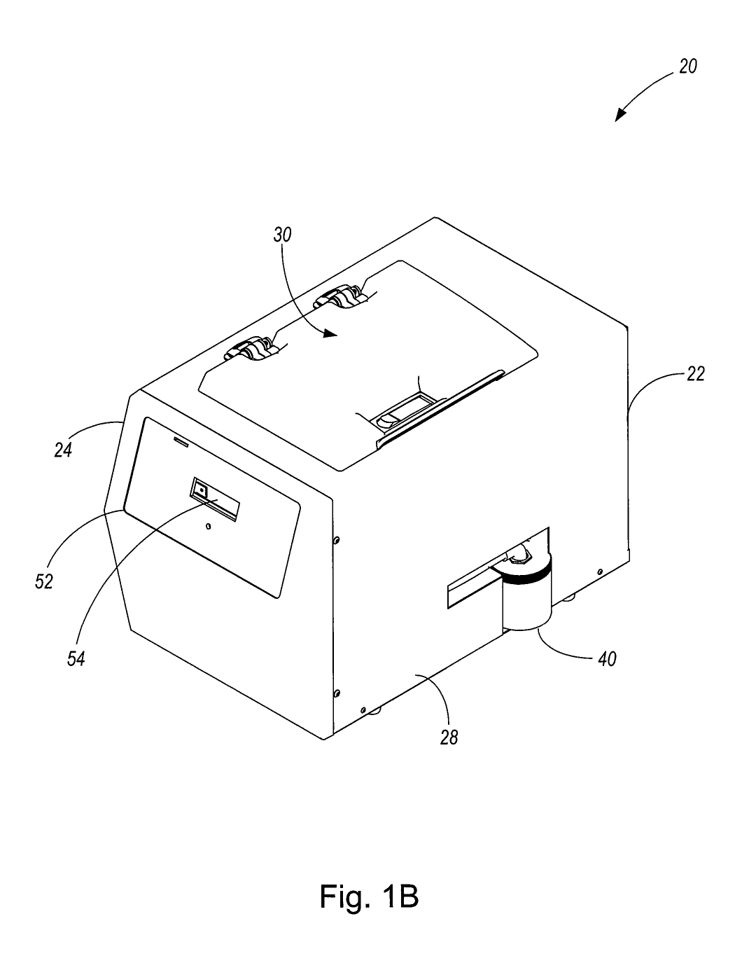

Referring now to the figures, an embodiment of a heat processing system 20 of this invention is shown in FIGS. 1A through 2B. FIGS. 1A and 1B are perspective views of system 20, FIG. 2A is a top view of system 20, and FIG. 2B is a cross-sectional view taken along line B-B of FIG. 2A. System 20 includes a body 22 with a front portion 24, a back portion 26, a side 28, a side 29, and a bottom 140.

A closure device 30 is provided on the top of system 20. Closure device 30 opens to allow access to a heat chamber 60 that is within body 22. A container of medical waste (such as canister 220) is placed into chamber 60 and treated as further described below. Closure device 30 has an upper portion 56 and a lower portion 58 that together make up the door or lid of the closure device. Mounting brackets 32 secure closure device 30 to the top of body 22. Mounting brackets 32 are pivotal or spring-like in nature to allow closure device 30 to be moved from open to closed positions. Other structures, such as sliding devices, caps, screw-on lids, etc. could be used as well to control access to chamber 60. A handle 36 and a lock 34 are also present on closure device 30. Lock 34 may be engaged to secure closure device 30 in the closed position, such as, for example, during operation of system 20. Body 22 is formed of any rigid material capable of withstanding the heat ranges and cycle times described herein, such as stainless steel sheet metal or plastics. In one embodiment, the material used is galvanized annealed steel. In one embodiment, body 22 has approximate dimensions of 23 inches deep by 16 inches wide by 13 inches high, but it should be understood that embodiments of systems of this invention may be any suitable size.

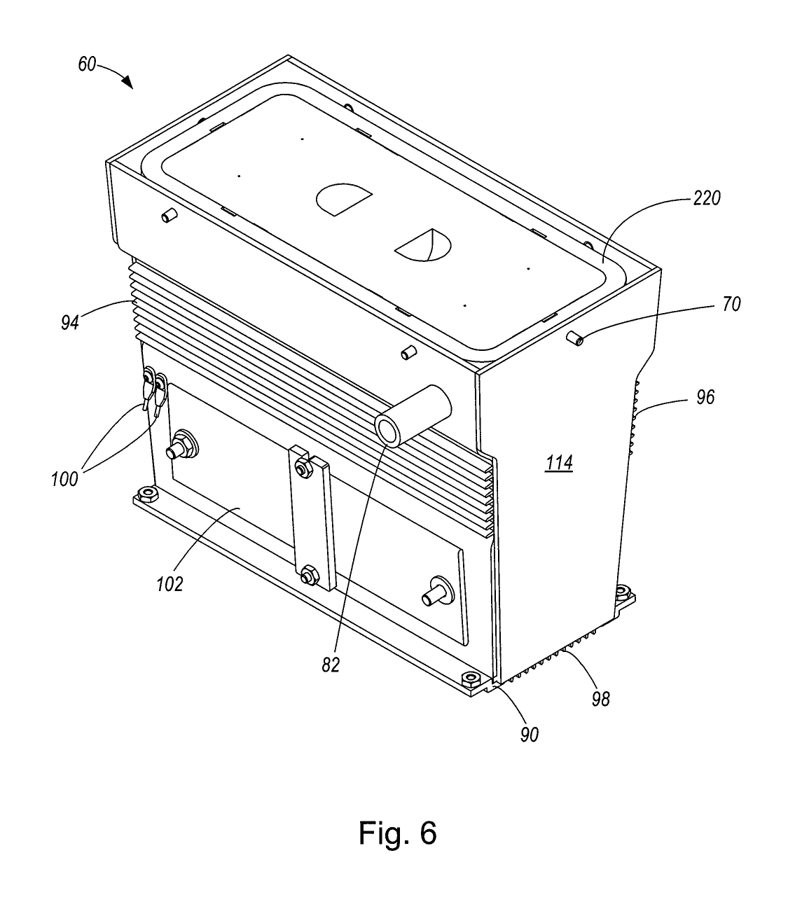

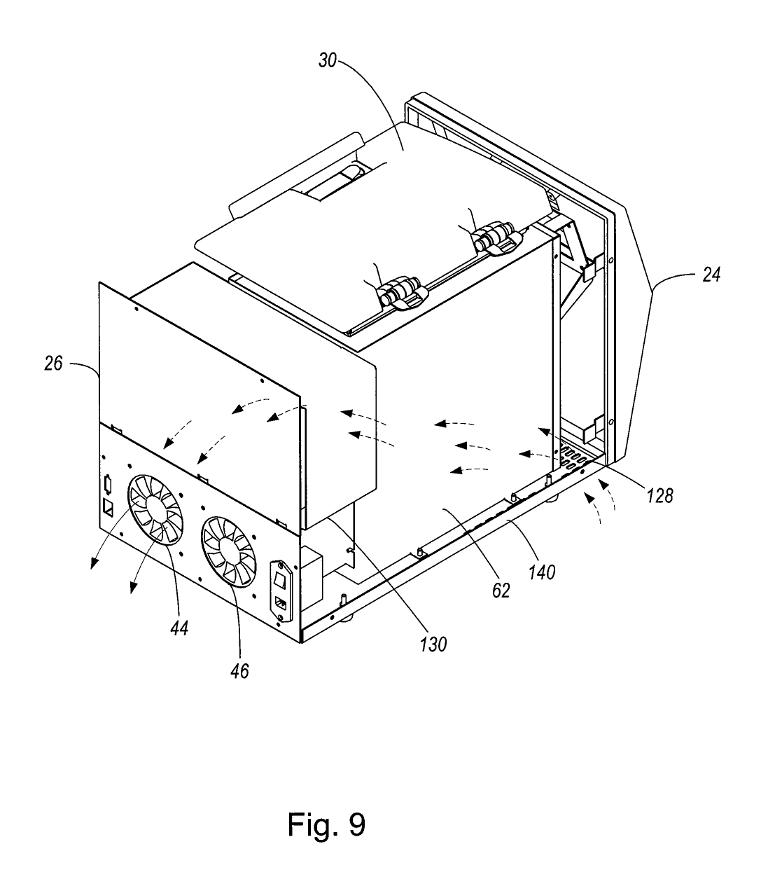

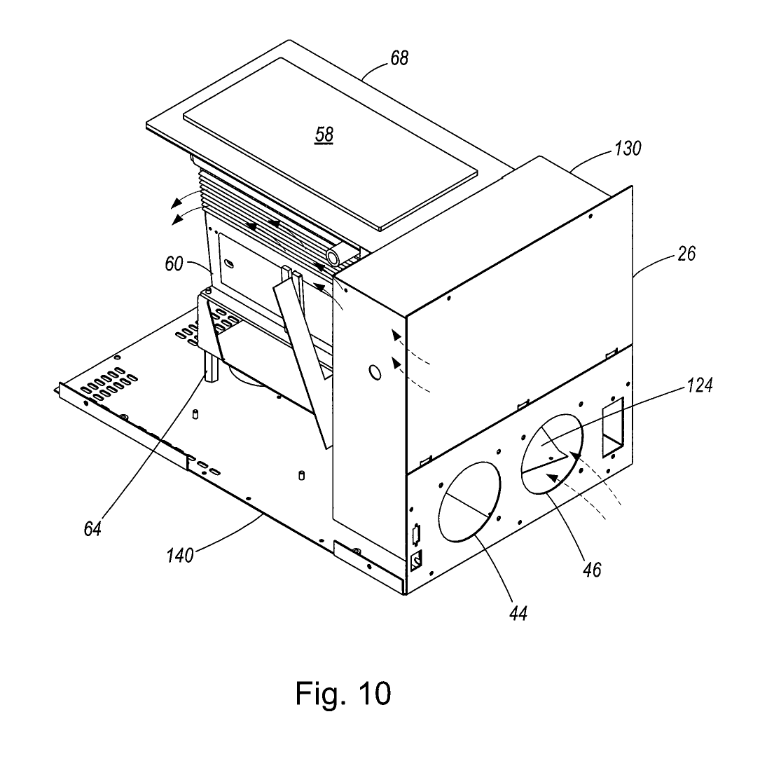

Side 28 includes a cavity 38 within which a tube 42 and ajar 40 are positioned. Tube 42 carries exhaust from chamber 60 in the form of liquid to jar 40, as is further described below with reference to FIG. 7. A pair of fans 44 and 46 are located on back portion 26 of system 20. Fan 44 cools the outer housing of body 22 and also provides negative air pressure which ensures air and gas flow through filter 78. Fan 44 is in continuous operation during use of system 20 and its use is described further below with reference to FIGS. 7-9. Fan 46 intakes outside air and drives the cooling air flow described below with reference to FIGS. 10-12. Suitable fans are commercially available from numerous manufacturers.

Back portion 26 also includes a standard power entry module 48, such as those found on most any commercially available personal computer, and a plurality of ports 50. In one embodiment, two ports are provided, one is for connection to a printer and one is a serial modem data port so that system 20 may transmit data remotely. In certain embodiments, system 20 has data storage and processing capabilities and allows for remote diagnostics and periodic process data transmission to a remote location to support data storage and system calibration and/or performance reviews, as further described below.

Front portion 24 of body 22 includes a display and control panel 52 with a window 54, as best seen in FIG. 1B. Panel 52 allows the user to control and monitor the operation of system 20. Much of the electronics that control system 20 are mounted on a bracket 72 inside body 22, as shown in FIG. 2B. A solid state relay 74 is also shown mounted to an exterior surface of a cage 62. As is well understood to those skilled in the art, system 20 will include electronics and circuits not shown in these figures that are necessary to control system 20 and components such as fans 44 and 46, heat sources for chamber 60, measurement devices that provide operational or other data, and the like. Some exemplary and suitable electronic and circuit components are fully described and shown in U.S. Pat. No. 5,972,291, while others will be apparent to those skilled in the art. It should be understood that modifications to those circuit components or others may be made.

Referring to FIG. 2B, chamber 60 is shown within body 22 of system 20. A container, such as a canister 220, is positioned within chamber 60. Chamber 60 is positioned within cage 62 inside body 22. Cage 62 is formed of any suitable rigid material capable of withstanding the heat ranges and cycle times described herein, and in one embodiment is made of galvanized annealed steel. In one embodiment, chamber 60 has approximate dimensions of 13 inches deep by 8 inches wide by 13 inches high, but it should be understood that embodiments of a chamber may be any suitable size.

A mounting bracket 64 is secured within cage 62, and a bottom 90 of chamber 60 is secured to mounting bracket 64. Mounting bracket 64 and an angled bracket 126 within cage 62 are also shown in FIGS. 7 and 10-13. Studs 66 protrude from the sides of cage 62 and hold insulation material (not shown) along interior portions of cage 62. A filter 78 is positioned within a filter housing 130 near back portion 26 of body 22. Filter housing 130 includes holes 80 in its exterior surfaces. Filter 78 is described further below with reference to FIG. 7.

A rim 68, which is best seen in FIGS. 7 and 10-12, sits upon a plurality of posts 70 that extend from near the top of chamber 60. When closure device 30 is closed, lower portion 58 of closure device 30 seals the top opening of chamber 60. The combination of rim 68 and lower portion 58 of closure device 30 ensures that no air flows in or out of chamber 60 during treatment of the waste, except through an exhaust tube 82. The flow of gas out of chamber 60 and into exhaust tube 82 is further described below with reference to FIG. 7. A thermal limiter 76 is coupled to chamber 60.

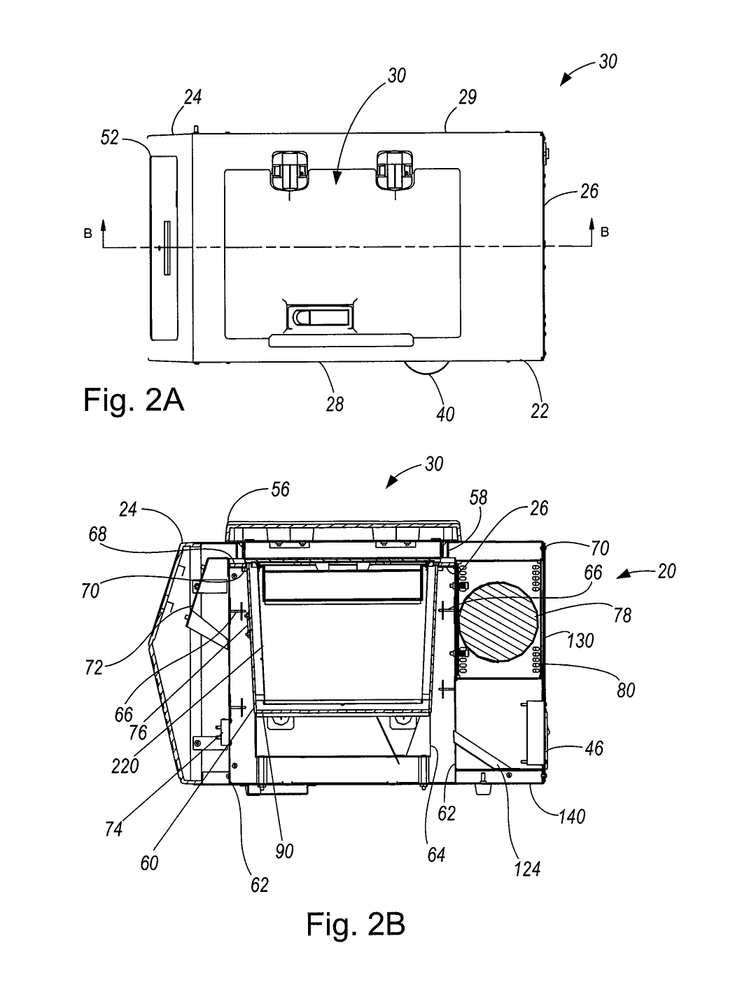

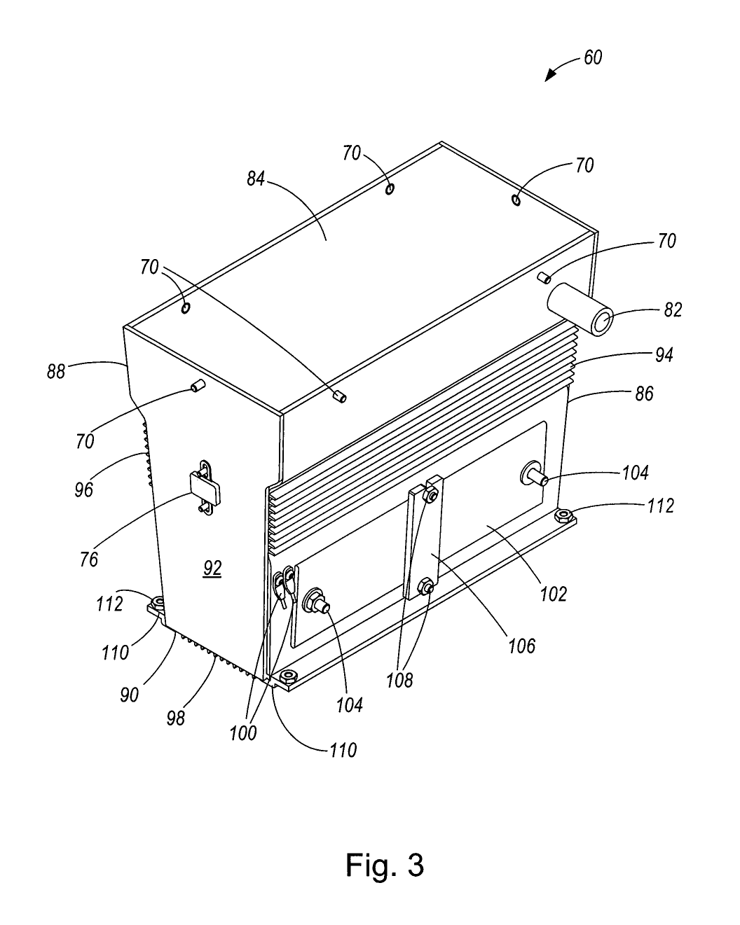

Referring now to FIGS. 3-6, heat chamber 60 of system 20 is described in more detail. FIGS. 3-6 are various views of chamber 60. Chamber 60 includes a plurality of posts 70 spaced around the exterior periphery of chamber 60. Posts 70 receive rim 68 as described above. Rim 68 surrounds the periphery of chamber 60 but does not cover an opening 84 at the top end of chamber 60 that receives a container of medical waste to be treated using system 20. Chamber 60 has long sides 86 and 88, a bottom 90, and short sides 92 and 114. In one embodiment, sides 86 and 88 and bottom 90 are extruded aluminum, sides 92 and 114 are cut or stamped pieces of aluminum, and sides 86 and 88, bottom 90, and sides 92 and 114 are welded together to form the central structure of chamber 60. In another embodiment, the entire chamber body may be cut or stamped from sheet aluminum, folded into shape and the seams welded. Bottom 90 includes flanges 110 extending generally transversely out from bottom 90. In one embodiment, flanges 110 are formed during extrusion of bottom 90. Chamber 60 is secured to mounting bracket 64 within cage 62 with fasteners (not shown) that extend through holes (not shown) formed in flanges 110 beneath nuts 112. In one embodiment, chamber 60 may be made in three pieces, with sides 86 and 88 and bottom 90 extruded as a unshaped channel and sides 92 and 114 fabricated and attached by welding or other means.

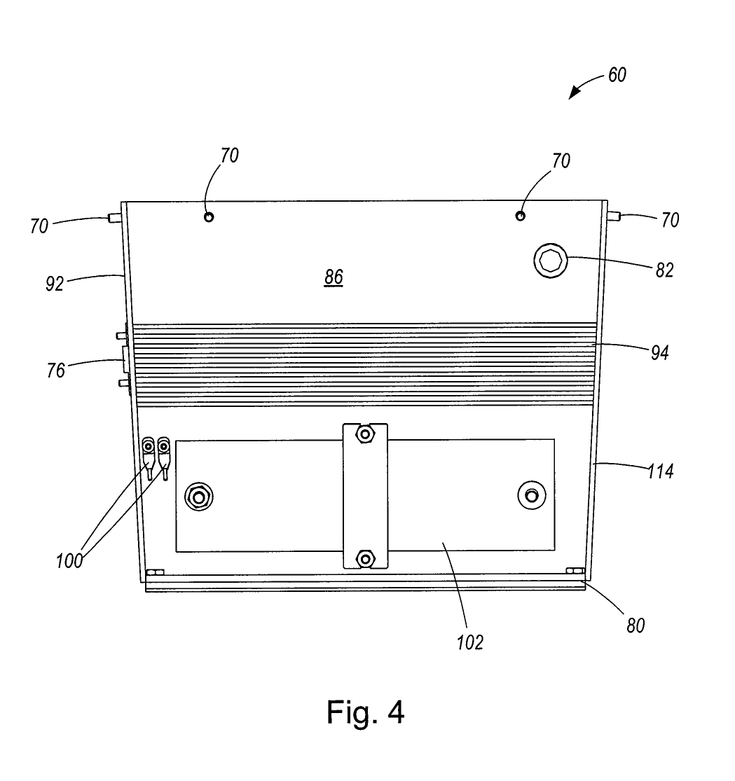

As seen best in FIGS. 3 and 5, sides 86 and 88 have a variable thickness and profile as they extend from opening 84 to bottom 90 of chamber 60. As shown in the figures, sides 86 and 88 increase in thickness at portions 116 and 118 where sides 86 and 88 contact plate heaters 102 and 122, respectively. This assists in optimizing heat transfer between plate heaters 102 and 122 and sides 86 and 88, respectively, of chamber 60. Thicker portions 116 and 118 allow heat to dissipate away from plate heaters 102 and 122 more effectively, preventing premature burn out of the plate heaters. Plate heaters 102 and 122 are heated up using any conventional method for directing power supplied to system 20 through power entry module 48 to components of the system, which results in the heating of the interior of chamber 60. Plate heater 102 is secured to the exterior surface of side 86 using threaded posts 104 and 108 and bar 106, and plate heater 122 is similarly secured to the exterior surface of side 88. In one embodiment, posts 104 and 108 are pressed into side 86 of chamber 60.

A plurality of fins 94 are formed on the exterior surface of side 86. Similarly, a plurality of fins 96 are formed on the exterior surface of side 88, and a plurality of fins 98 are formed on the exterior surface of bottom 90. In one embodiment, the sets of fins 94, 96, and 98 are formed in sides 86 and 88 and bottom 90 during extrusion. In the folded embodiment of the chamber, extruded finned aluminum may be mechanically joined to the sides and/or bottom of the chamber. Fins 94, 96, and 98 are incorporated into chamber 60 to assist in cooling the chamber rapidly after heat processing of a container of medical waste is completed. Cooling air flow through system 20 is further described below with reference to FIGS. 10-12. Exhaust tube 82 is secured to side 86 of chamber 60. Exhaust tube 82 allows expanding gases to exit chamber 60, but air is not otherwise coming into or being drawn from chamber 60 through exhaust tube 82. Exhaust tube is further described below with reference to FIG. 7.

A thermal limiter 76 is mounted or coupled to side 92. Thermal limiter switches are well known to those skilled in the art, and thermal limiter 76 is provided to turn off plate heaters 102 and 122 in the event chamber 60 becomes overheated. Thermocouples 100 are disposed on side 86 and thermocouple 120 is disposed on side 88. Thermocouples 100 and 120 measure the temperature of the exterior surfaces of those sides of chamber 60. Plate heaters 102 and 122 are electrically connected to power through thermal limiter 76. If the temperature of chamber 60 at the location of thermal limiter 76 exceeds a designed set point of thermal limiter 76, thermal limiter 76 will break the power circuit to plate heaters 102 and 122, shutting them down to prevent overheating of system 20.

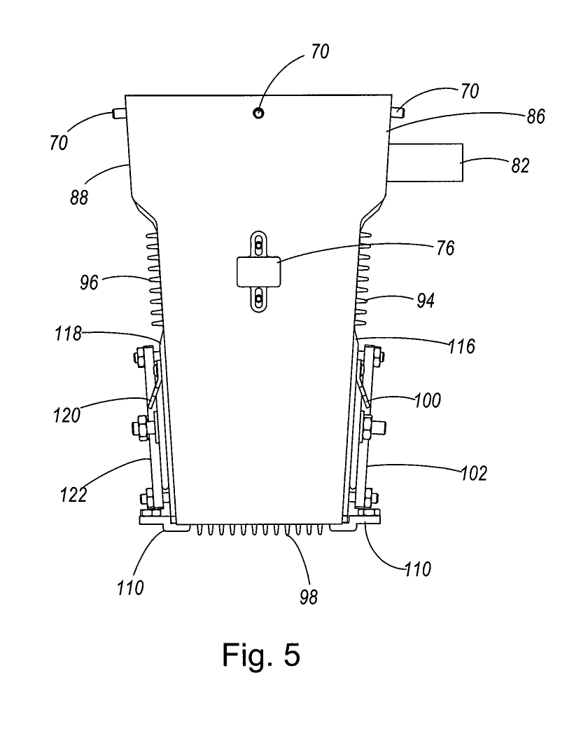

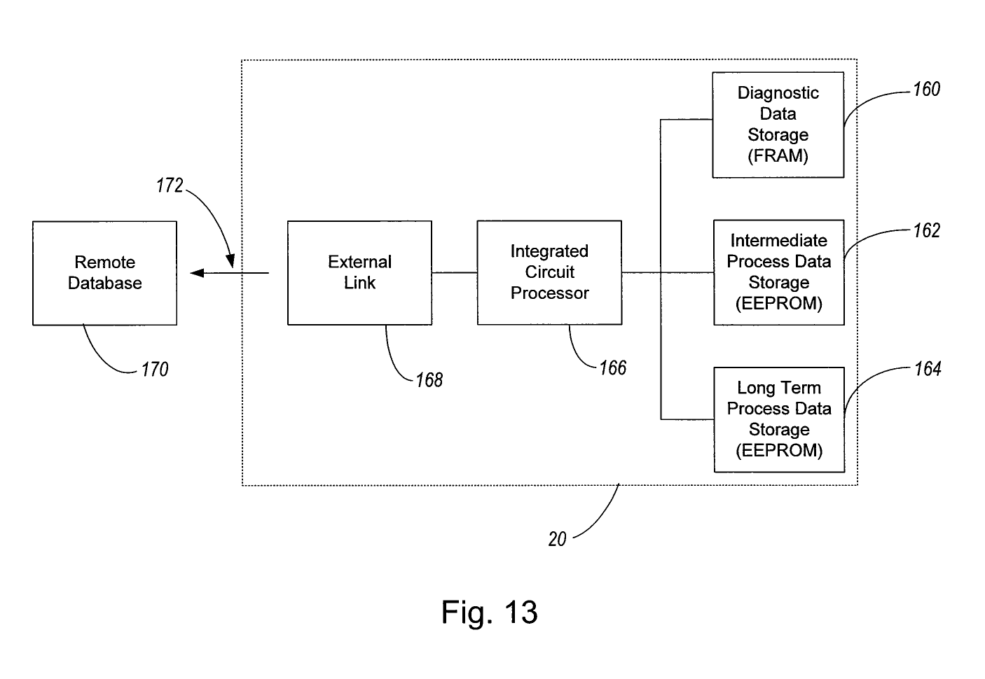

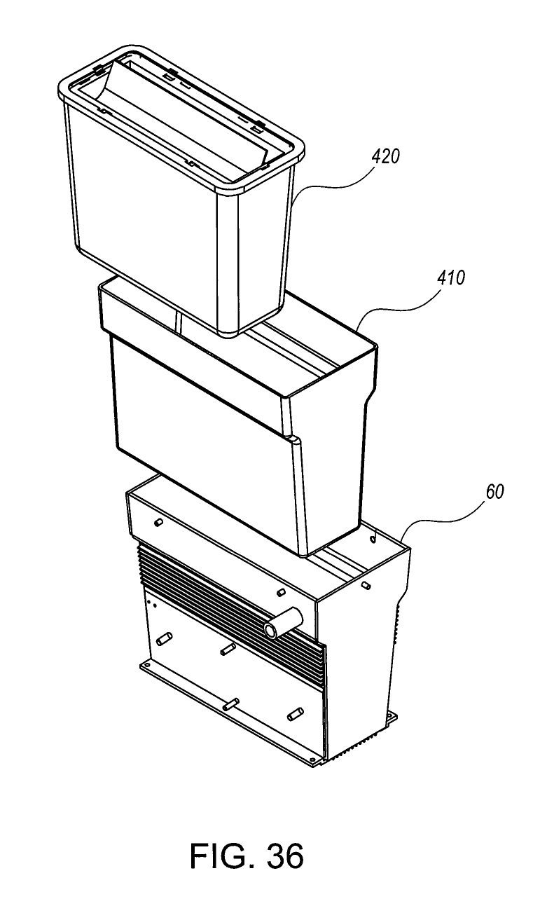

A container of waste, such as canister 220 shown in FIGS. 2B and 6 (as well as FIGS. 14, 17, 19, 25, 26 and 28), is placed into chamber 60 when closure device 30 is in the open position. The sides of container 220 contact the interior surfaces of sides of chamber 60 in the area where plate heaters 102 and 122 are coupled to chamber 60. In other embodiments, chamber 60 may be slightly larger than the container of waste that it receives such that a very minimal open space exists between the container and all interior surfaces of chamber 60. In other embodiments, a sleeve 410 (as shown in FIG. 36) may be reusable and placed inside chamber 60, and a canister 420 designed to melt may be placed within sleeve 410, such that the sides of sleeve 410 contact the interior surfaces of chamber 60.

Once the container is in position, closure device 30 is closed and lock 34 is engaged. A lower portion 58 of closure device 30 extends within body 22 and effectively seals the top opening 84 of chamber 60. There is no air flow in or out of chamber 60, except through exhaust tube 82, as described above and described further below. As plate heaters 102 and 122 are brought to the proper temperatures for sterilization and/or to render the waste material unrecognizable and unreusable, a user of system 20 can monitor the container through the display and control panel 52 on front portion 24 of body 22 of system 20.

In certain embodiments, a typical process cycle time for a container of medical waste is approximately two hours to two and one-half hours. The system takes approximately 18 minutes to heat from room temperature to a temperature in the desired range of about 300 to about 425 degrees Fahrenheit, after the container is placed in the heat chamber of the system. After the chamber reaches the desired temperature, the container of waste is held and heated at this temperature for about 60-90 minutes. The system is maintained in the closed and locked position as the medical waste is heat processed. The container is then allowed to cool to a safe handling temperature of approximately 120 degrees Fahrenheit or less before the container can be removed. This cool down takes about 30 minutes. It should be understood that embodiments of systems and methods of this invention are not limited to the cycle times disclosed above, which are merely exemplary.

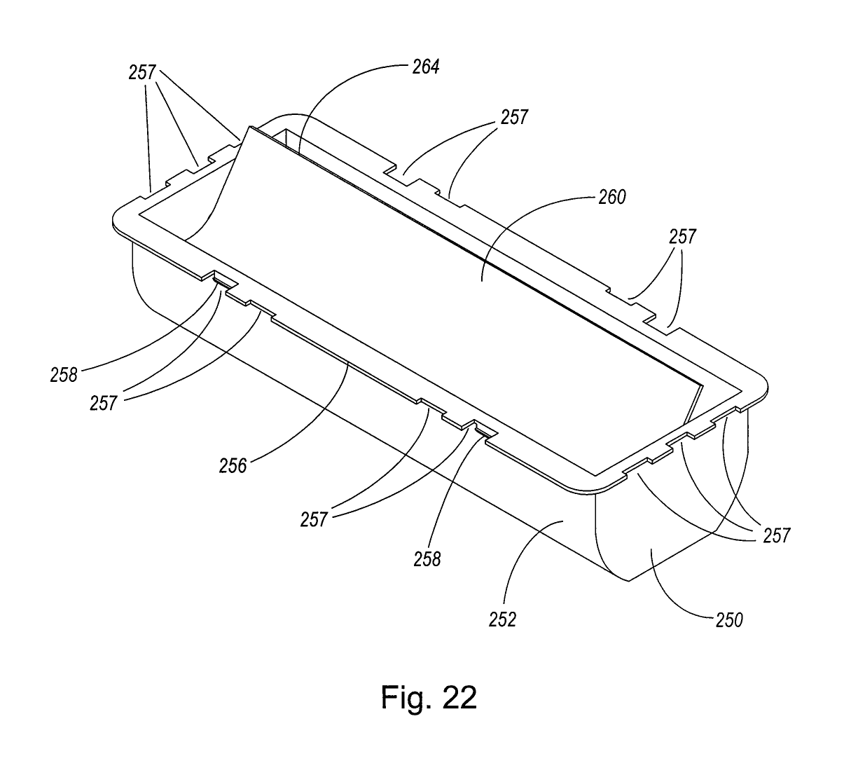

As shown in FIG. 6, canister 220 is placed within chamber 60 such that the waste within canister 220 is heated to sterilize the waste and render any sharps material non-recognizable and non-reusable. As shown, the container is a canister and rim combination sealed with a plug, such as will be discussed below with respect to FIGS. 14-27. When used with this container, at least a portion of the interior surfaces of sides 86 and 88 of chamber 60 contact the sides of the canister that have a complementary shape, providing for direct thermal conduction between sides 86 and 88 of chamber 60 and the sides of the canister. This is advantageous in comparison to existing systems that relied on thermal radiation to treat the waste in the container because the sides of the heat chamber did not contact the sides of the container.

Nevertheless, it should be understood that containers of numerous other shapes and sizes may be used within chamber 60 and the waste therein treated accordingly; provided, of course, that such containers fit sufficiently within chamber 60 so that the system may be closed and locked during the heat treatment. Moreover, containers may be made of numerous shapes, such that one or more side surfaces of the container contacts the side surfaces of chamber 60, particularly those sides to which plate heaters or other heat sources are mounted. Additionally, containers that have an exposed collection opening, in contrast to those sealed with a plug or the like as is canister 220, may also be used within chamber 60. In such instances, the hardened mass that results after the warping or melting of any plastic material that is part of the waste within the container is preferably larger than the collection opening of the container, thereby aiding in the prevention of the removal of the mass from the container.

As mentioned above, in some embodiments, a canister 420, as shown in FIG. 36, may be fabricated from a thermoplastic material that melts and encapsulates the medical waste upon the application of dry heat. When such a canister is used, a sleeve 410 may be used, rather than placing canister 420 directly into chamber 60. Sleeve 410 may be constructed of aluminum and the interior coated with a non-stick material. However, this embodiment is not limited to such materials; any material that is suitable for repeated usage and removal of processed, melted medical waste canisters may be used. When such an embodiment is utilized, canister 420 is inserted into sleeve 410, and the combination is then inserted into chamber 60 where the dry heat is applied. Upon cooling down, sleeve 410 is removed from chamber 60, and the processed contents, formally canister 420 and waste within, may be discharged.

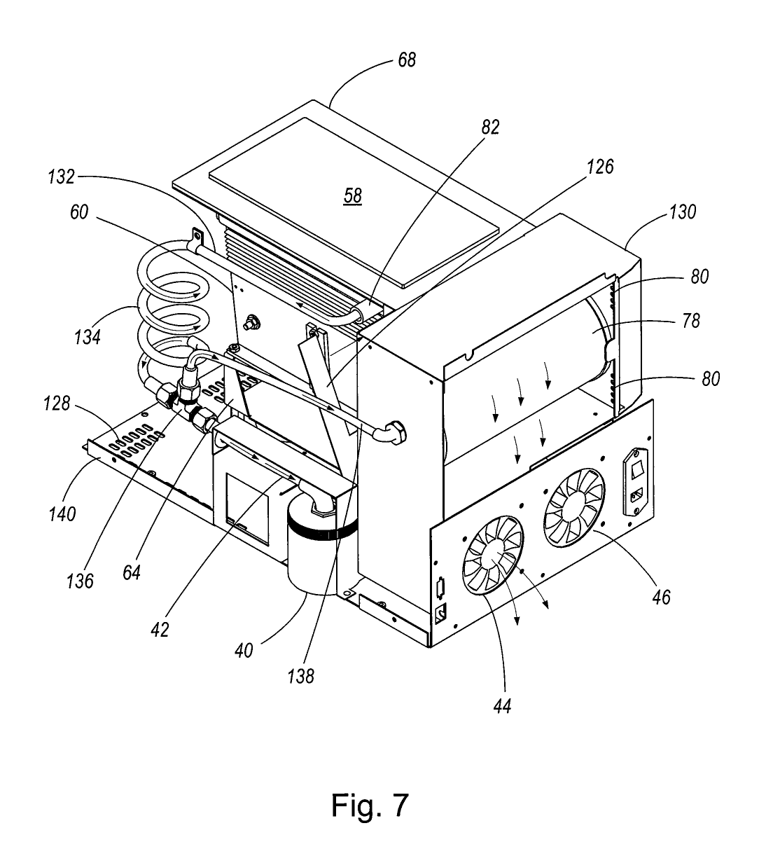

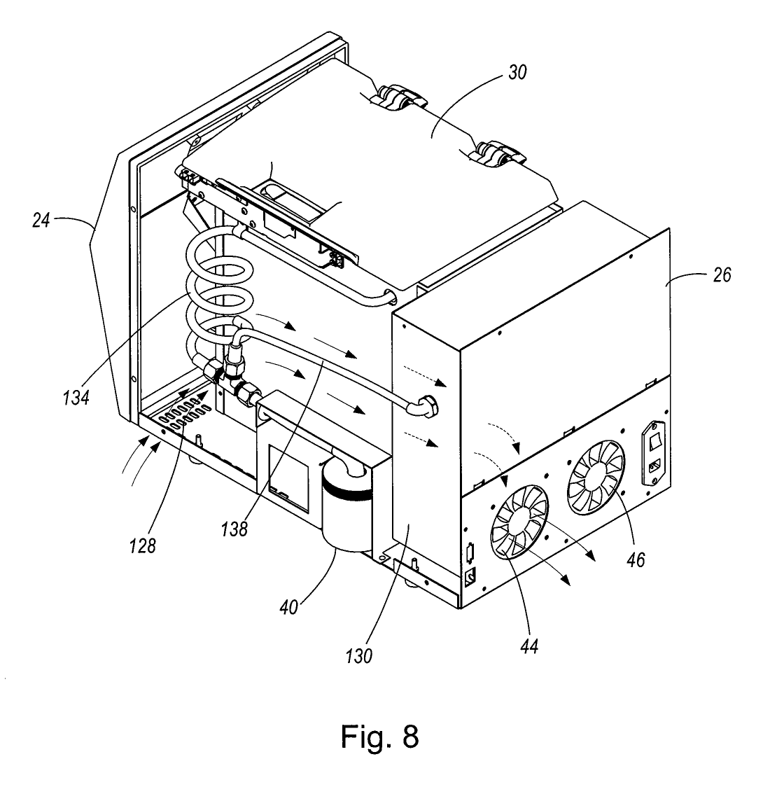

Turning back to operation of system 20, there are three controlled air flows in certain embodiments of systems and methods of this invention: a heat chamber exhaust flow, ventilation air flow, and cooling air flow. An exemplary heat chamber exhaust flow is shown in FIG. 7, an exemplary ventilation air flow is shown in FIGS. 8 and 9, and an exemplary cooling air flow is shown in FIGS. 10-12. The various perspective views in FIGS. 7-12 show parts of interior portions of an embodiment of heat processing system 20, with some components removed or not shown so that the various air flows can be clearly illustrated. The various air flows operate to handle exhaust from the heat chamber 60, ventilate and cool interior components of system 20, and prevent the discharge of hazardous or bio-hazardous fumes from system 20 as waste material is heated and sterilized.

The heat chamber exhaust flow, or exhaust flow, is shown in FIG. 7. This flow provides an escape for expanding gases as the container with waste is heated within chamber 60 and such outgassing products are created. There is no air coming into chamber 60, and there is no mechanism withdrawing air from chamber 60. It should be understood that when medical waste, or any waste for that matter, comprised of different materials is heated, there is typically no orderly heating of the material. The waste material does not undergo an even or uniform heating, as there are different compositions and localized volumes within the container being heated. For example, if a pocket of alcohol is heated within the medical waste, it will start to out gas before the surrounding materials because of the low vapor point of the alcohol. This will produce a rapid and voluminous flow of gas. Due to this random heating, system 20 is designed to handle the associated unpredictable heating and gas expansion problems that may occur.

Exhaust tube 82 is secured to side 86 of chamber 60. Exhaust tube 82 may be made of aluminum and welded to side 86 of the chamber 60. The outgassing products exit chamber 60 through exhaust tube 82 and move into tube 132 as they expand. Tube 132 provides a safety volume of space for gas from chamber 60 which occurs upon heating medical waste in a container placed within chamber 60. Tube 132 is preferably made of a metal, copper for example, that can withstand holding gases at high temperatures.

Tube 132 includes a spiral portion 134 that ends at a T-valve 136. Tube 132, and particularly spiral portion 134, acts as a condensation tube assisted by air flow that passes by tube 132, as shown in FIGS. 8-12. Accordingly, the contents within tube 132, including spiral portion 134, are being cooled such that “dry” gas and condensed water vapor are within the tube when it ends at T-valve 136. The gas passes up through T-valve 136 and into a tube 138 that carries the gas to filter 78. Tube 138 may be made of a suitable plastic, such as nylon, or other material. The gas flows through filter 78 and then out of system through fan 44, as shown in FIG. 7. Filter housing 130 includes numerous holes 80 in its surfaces and is configured to draw enough air past filter 78 to create a negative air pressure therein that helps ensure that all exhaust gas passes through filter 78.

The condensed liquid passes through T-valve 136 and tube 42 where it collects in jar 40. Tube 42 may be made of a suitable plastic, such as nylon, or other material. Jar 40 is sealed, and in one embodiment has a capacity of about eight ounces. At such a capacity, jar 40 will typically need to be emptied by an operator and replaced only after numerous cycles. The separation of moisture from the heat chamber exhaust improves the life and efficiency of filter 78, as reducing the exposure of filter 78 to moisture lengthens the useful life of filter 78.

Filter 78 is preferably an odor-trapping filter coupled with an air filter material capable of filtering particles potentially contaminated with viruses and microbials. An example of a suitable filter is an electrostatically-charged air filter medium. In one embodiment, filter 78 is a dual stage charcoal type filter. Gas passes through the air particle/biological portion of filter 78 first and then passes through a charcoal portion of filter 78. For maximum effectiveness, the exhaust to be filtered should pass over and around the charcoal slowly enough for the charcoal to trap and absorb the odors.

FIGS. 8 and 9 show ventilation air flow through system 20. Air is drawn in through the plurality of holes 128 formed in bottom 140 of body 22 of system 20. Similar holes may also be provided in lower portions of the sides of body 22 of system 20. The air is drawn past tube 132, including spiral portion 134 on one side of system 20, as shown in FIG. 8, and along the outside of cage 62 on the other side of system 20, as shown in FIG. 9. The air moves through the rear end of system 20, including the interior portion of filter housing 130. The air does not pass through filter 78, but around it, creating the negative pressure created in that area as it is drawn down through fan 44 and out of body 22 of system 20. The ventilation air flow helps maintain the outer surfaces of body 22 at low temperatures, as well as providing adequate ventilation to the interior components of system 20.

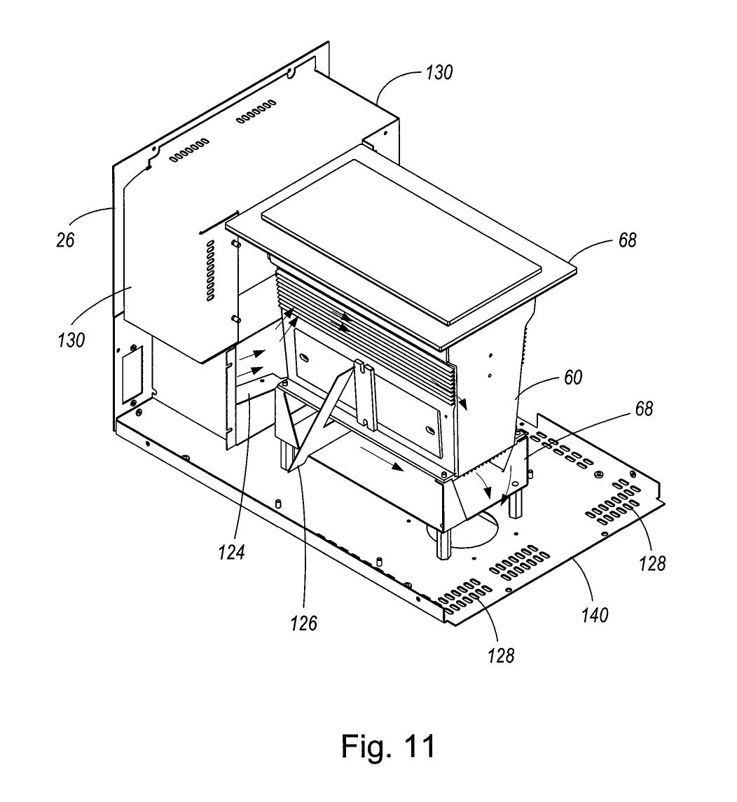

FIGS. 10-12 show cooling air flow through system 20. This air flow is created by fan 46 bringing in outside air that is directed into the interior of system 20 using a deflector 124. The air flows up and along the sides and bottom of chamber 60, bringing air past the plurality of fins on the sides and bottom of chamber 60 to assist in cooling chamber 60. The air then flows down through an opening 142 in bottom 140 of body 22 of system 20. A bracket 144 is mounted beneath opening 142 and directs the air out to the sides as shown in FIG. 12.

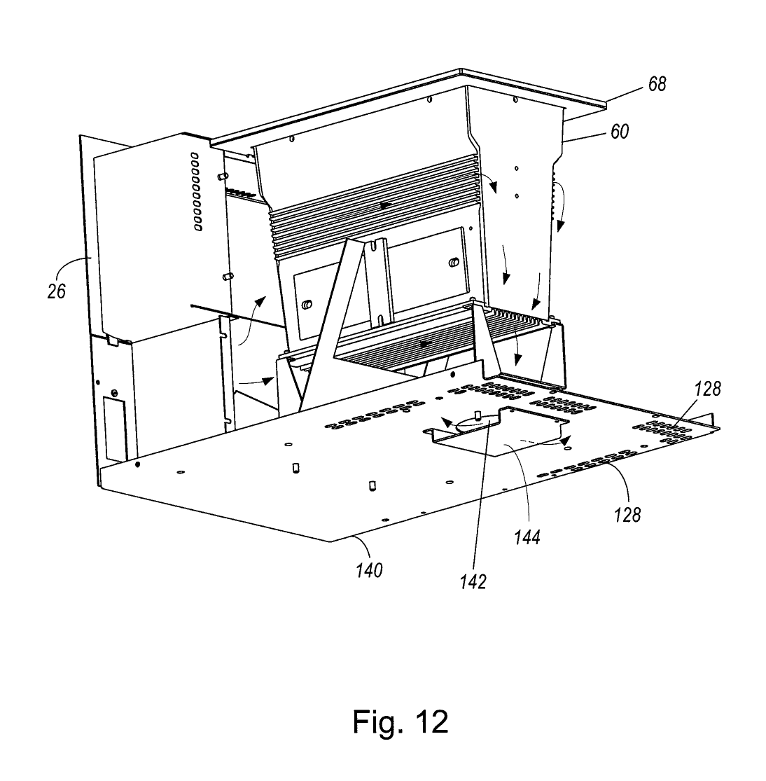

As indicated above, certain embodiments of system 20 may have data storage and processing capabilities and allow for continuous monitoring of critical process parameters, remote diagnostics and periodic process data transmission to a remote location to support data storage and system calibration and/or quality performance reviews. In one embodiment, system 20 is equipped with an external link 168, a processor 166, and three data storage components 160, 162, and 164, as shown in FIG. 13. System 20 may contact or be contacted by a remote server or database 170 via a wireline, wireless, or similar connection 172 through external link 168. Components 160, 162, and 164 are EEPROM or FRAM components well known to those skilled in the art and support data management, analysis, and reporting functions. Data management and reporting software is integrated into the system to support continuous evaluation of critical operating parameters and periodic quality control and system performance analysis and to allow for periodic uploading of process run data to a remote site, uploading of diagnostic data in the event of a process failure to a remote site, and download of periodic firmware upgrades remotely.

A system may through firmware have a “FAIL SAFE” whereby a treatment is successful only if the heat chamber containing the waste load has heated to at least a minimum treatment temperature, held at or above this treatment temperature for a minimum process time, cooled to a safe handling temperature (typically about 120 degrees Fahrenheit or less), and process certification labels have been printed through use of a periphery serial label printer.

During a treatment cycle, critical system states may be recorded including the measurements of each of the thermocouples, the state of the door latch and the locking bolt, the states of the fans, time, the temperature of the electronic printed wire board assembly (PWBA), and the state of various process flags that note the progress through the major cycle components (heating, treating, cooling, printing). These states are sampled periodically (e.g., every minute, every five minutes, etc.) and stored in a data ring buffer.

In addition to the above information, a consecutively assigned run number may be assigned to each process. As part of a user system interface, the operator may be prompted to enter the type of waste to be treated (i.e., sharps or red bag waste). Various other data is recorded for each process run including the serial number of the machine, the date, the times for process start, treatment start, treatment end and cooling end, minimum and maximum heat chamber temperature recorded during the treatment phase of the cycle, the temperature control set point for the heat chamber, the minimum regulatory treatment temperature, and the minimum regulatory process time.

Three primary data buffers may be present and managed with system firmware. A diagnostic buffer, such as component 160, contains the most information with sampling of all measurable system states every minute from the start of the process through the completion of the cooling cycle. This data buffer is capable of holding a comprehensive data set for the most recent 5-10 treatment cycles. In the event of a system failure, the system may automatically contact a remote database through a modem or other external link to upload this critical process information for troubleshooting purposes. An intermediate data buffer, such as component 162, contains critical process data including the run number, date, and the measurement of at least one of the thermocouples on a periodic basis through the treatment cycle. The size of this ring buffer typically allows storage of at least thirty days of process data for the typical user of the system. A long term data buffer, such as component 164, includes a longer term data set that records critical process data information for each process run including run number, date, times for process start, treatment start, treatment end, and cooling end, the minimum and maximum temperatures observed through monitoring of the thermocouples during the treatment cycle, and the type of waste treated.

On a periodic, typically monthly basis, a system with the above capability may remotely contact a system database using either a built-in modem or other means. All three data buffers are uploaded upon successful connection with the remote database. The extensive diagnostic buffer is used to perform periodic system performance/quality control monitoring to ensure that all systems are functioning within appropriate specifications and tolerances. Data from the intermediate and long term data buffers are permanently stored in the remote database as a back-up system to support regulatory compliance documentation.

Turning now to one embodiment of a canister for use within chamber 60 of a heat processing system of this invention, a canister 220 is shown in FIG. 14. Canister 220 is slightly tapered from its top end to its bottom and has a seamed bottom and a rolled lip 222 at its top end. A plurality of slots 224 are just beneath rolled lip 222 to facilitate attachment of a rim, lid, or other top, such as rim 230 described below. Although canister 220 is generally of a rectangular shape and is tapered, it should be understood that other shapes, including non-tapered designs, are suitable for canisters in accordance with this invention. In one embodiment, canister 220 is made of tin, which can withstand a dry heat sterilization cycle of up to about 425 degrees Fahrenheit for up to about one-hundred twenty minutes and maintain its dimensional stability. If canister 220 is to be used with an apparatus for heat processing of medical waste, canister 220 may be made of any suitable material that is able to maintain its dimensional stability at dry heat sterilization temperatures of up to about 425 degrees Fahrenheit, including plastics. In one embodiment, canister 220 may have approximate dimensions of 10 inches deep by 4 inches wide at its top and 9 inches deep and 3 inches wide at its bottom, and 8 inches high, but it should be understood that embodiments of a canister may be any suitable size.

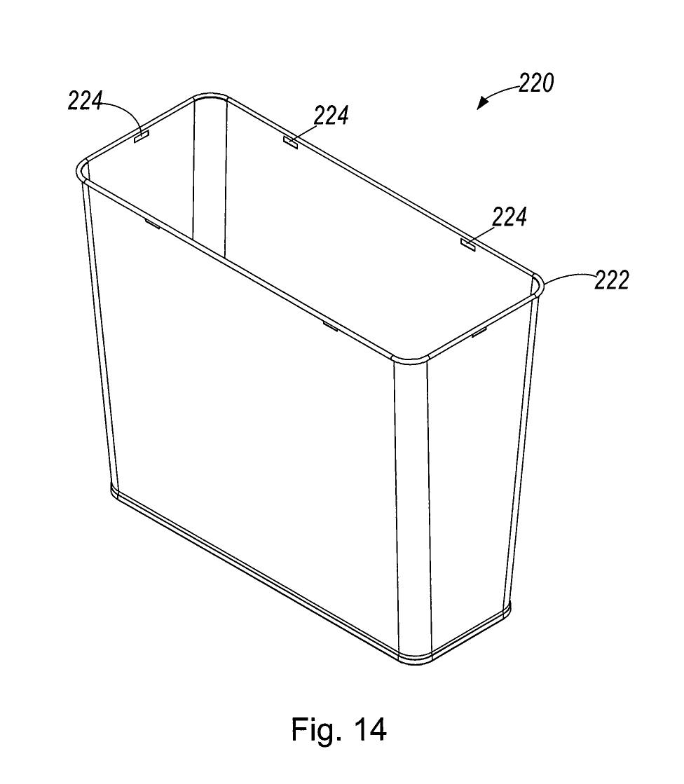

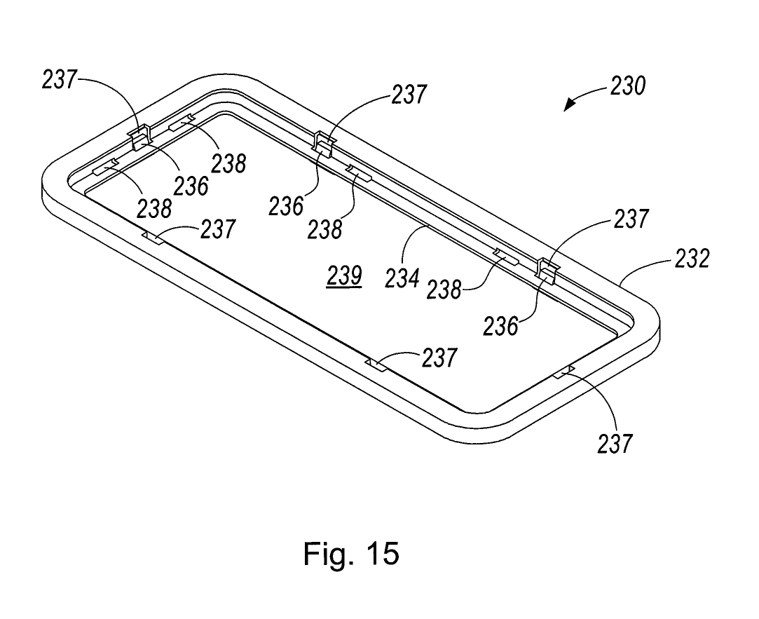

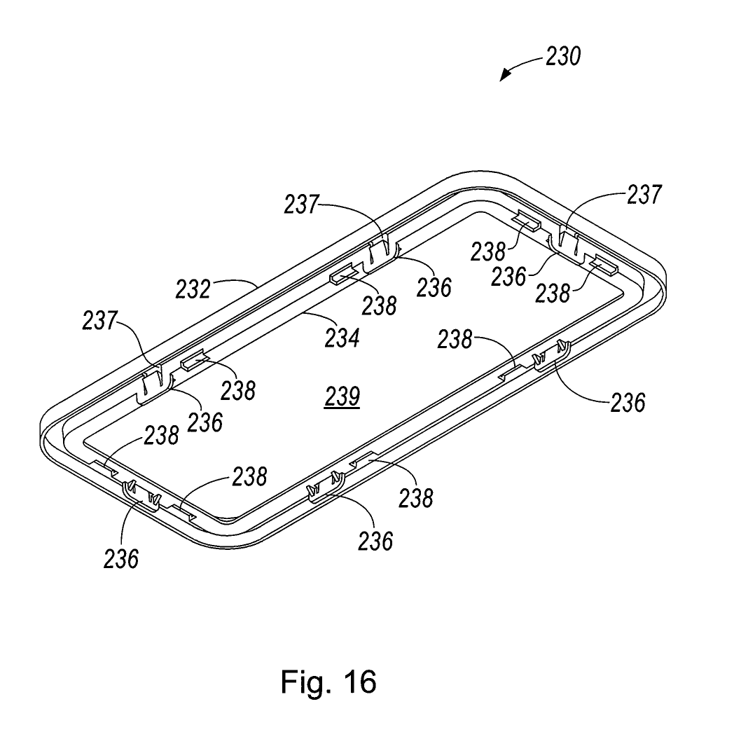

In an embodiment used for red bag waste collection and processing, canister 220 is used with rim 230 and a plug 240. As shown in FIGS. 15 and 16, rim 230 includes an outer portion 232 and an inner portion 234. Outer portion 232 of rim 230 fits over rolled lip 222 of canister 220, as shown in FIG. 17. Projections 236 are spaced around the periphery of outer portion 232 and extend downward from outer portion 232. A small aperture 237 is formed above just above each projection 236. Projections 236 engage slots 224 of canister from the interior of canister 220 to secure rim 230 to canister 220. The use of rim 230, and the engagement of projections 236 into slots 224 from the interior of the canister, significantly reinforces the top end of canister 220, which is useful, for example, when a thin tin material is used to make canister 220. Additionally, the manner of engagement of rim 230 over rolled lip 222 of canister 220 decreases the significance of any variance in the dimension of rolled lip 222 that may occur during manufacturing of canister 220.

An inner portion 234, which includes a plurality of slots 238, is seated in the interior of the canister opening upon engagement of rim 230 and canister 220. Rim 230 includes a large, central opening 239, through which infectious and medical waste may be disposed into canister 220. For use with a heat treatment process, such as those described above, rim 230 and plug 240 may be made of any suitable material that can maintain its dimensional stability in dry heat temperatures of up to about 425 degrees Fahrenheit. In a preferred embodiment, rim 230 and plug 240 are made of Nylon 6/6. Rim 230 and plug 240 may also be made of Nylon 6/6 plus glass fiber, polyethlyene terephthalate (PET), PET plus glass fiber, polyetheretherketone (PEEK), polyetherimide (PEI), Teflon or polytetraflouroethylene, or other materials.

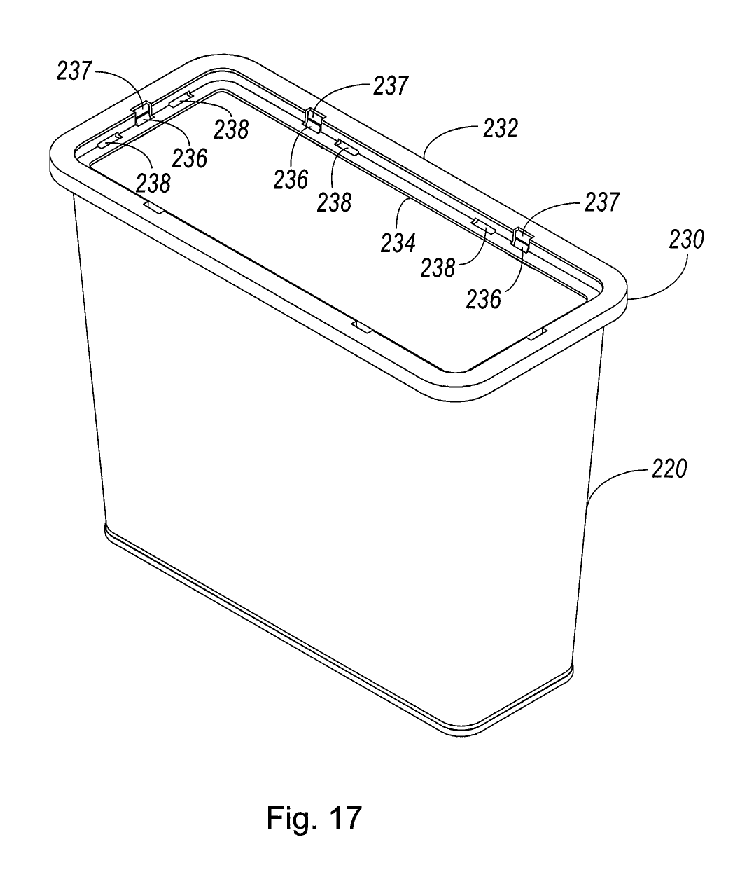

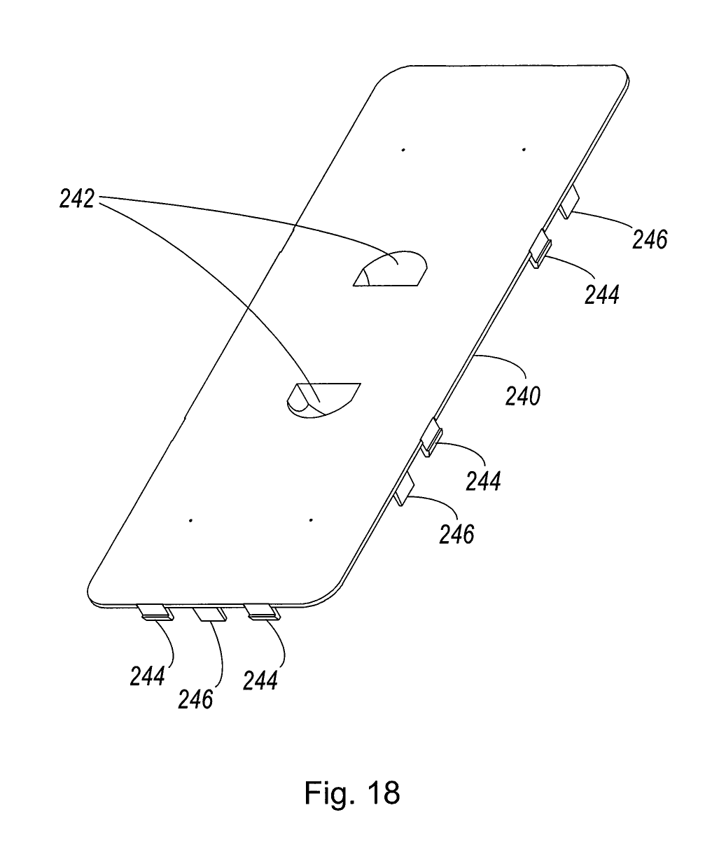

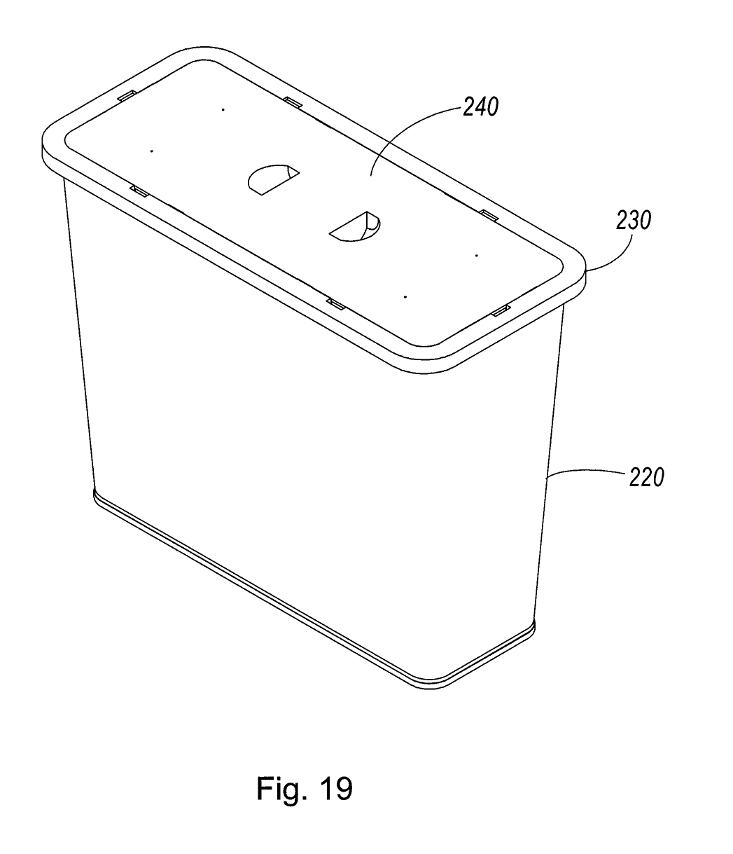

Plug 240 is shown in isolation in FIG. 18 and assembled with canister 220 and rim 230 in FIG. 19. Additionally, FIG. 20 shows a perspective view from underneath assembled rim 230 and plug 240. Once canister 220 is filled with waste and ready for heat processing or other disposal, plug 240 is inserted into rim 230. Depressions 242 are formed in the top of plug 240. When used in conjunction with a heat processing apparatus, for example, depressions 242 allow a person to easily and efficiently place the canister/rim/plug combination into the heat chamber and to remove it from the heat chamber after it has been processed. A plurality of hooks 244 are spaced around the periphery of plug 240. Hooks 244 snap fit into slots 238 in rim 230 to positively engage rim 230 and plug 240.

Because canister 220 is used to hold infectious and medical waste, it is desirable that the contents of heat-processed canisters be difficult to access after collection of the waste and through treatment and final disposal. In other words, it is desirable that a lid system, such as rim 230 and plug 240, for example, or rim 230, plug 240, a chute, and a movable member (described below), placed atop canister 220 should be difficult to remove once the canister has been sealed. Because rim 230 engages canister 220 from its interior, projections 236 extend through slots 224 and are accessible from the outside of canister 220. Accordingly, in a preferred embodiment, plug 240 includes a plurality of tabs 246 spaced around the periphery of plug 240. Upon engagement of rim 230 and plug 240, tabs 246 backfill apertures 237 of rim 230, as is best shown in FIG. 20. The receipt of tabs 246 within apertures 237 keeps projections 236 of rim 230 from being easily backed out of slots 224 of canister 220, thereby preventing disengagement of rim 230 and canister 220 and keeping the plug/rim/canister combination sealed and tamper-resistant.

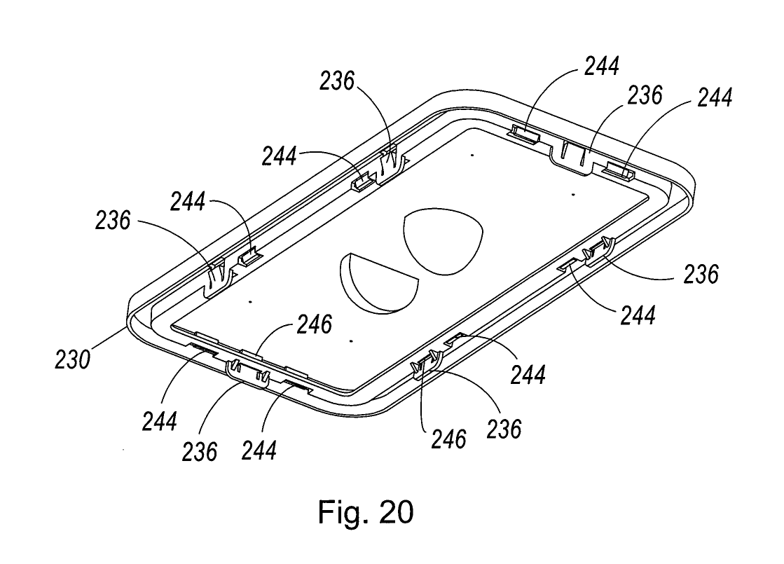

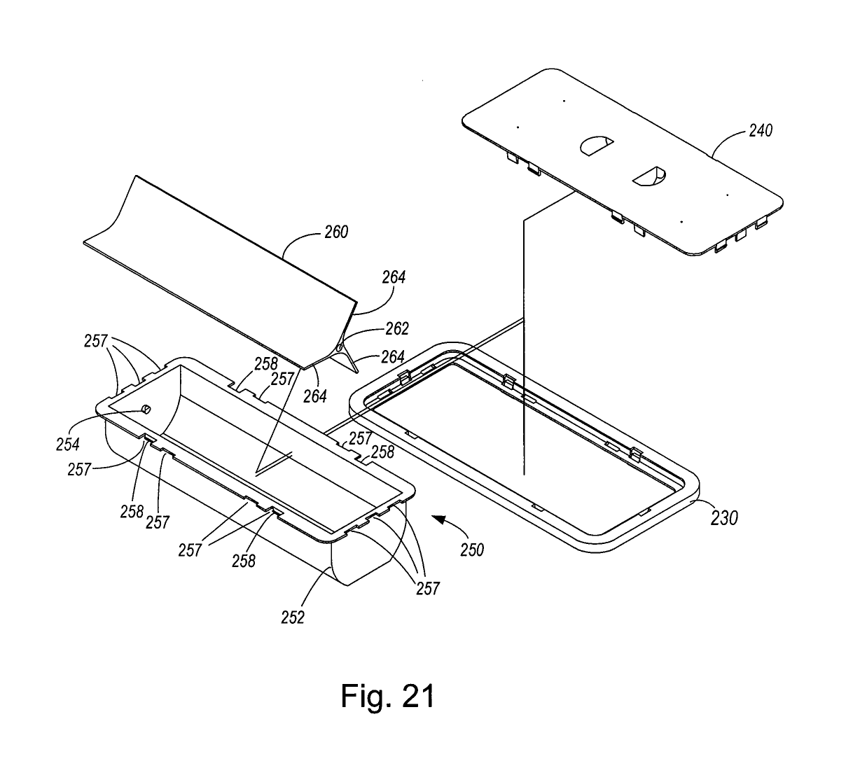

Certain embodiments for use in collection of sharps material comprise other components configured to provide for safe loading of sharps material in addition to the canister, rim, and plug components described above. In one embodiment, an assembly of a chute 250 and a spinner 260 are used in combination with canister 220 and rim 230. Chute 250, spinner 260, rim 230, and plug 240, which is used once canister 220 is full and ready to be disposed of or processed, are shown in the perspective, exploded view of FIG. 21. FIG. 22 shows a perspective view of chute 250 and spinner 260 assembled.

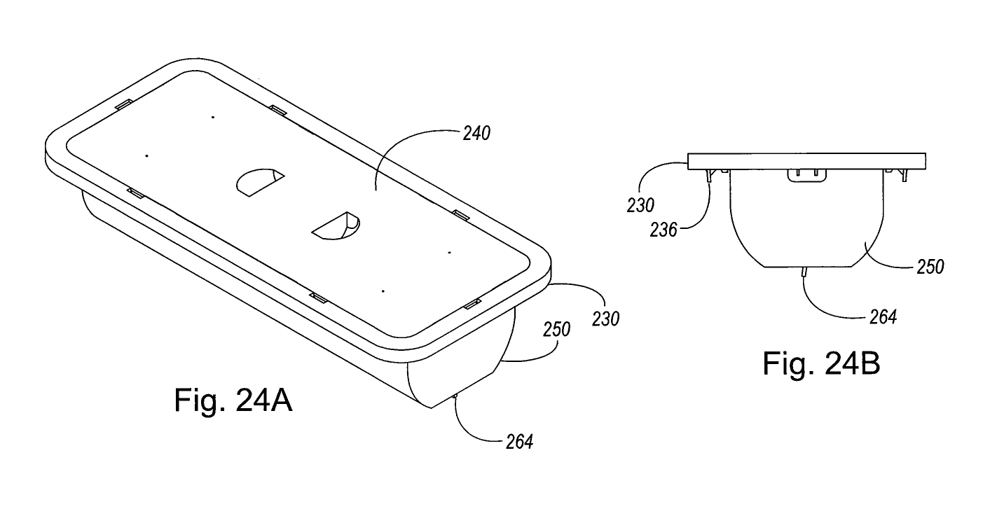

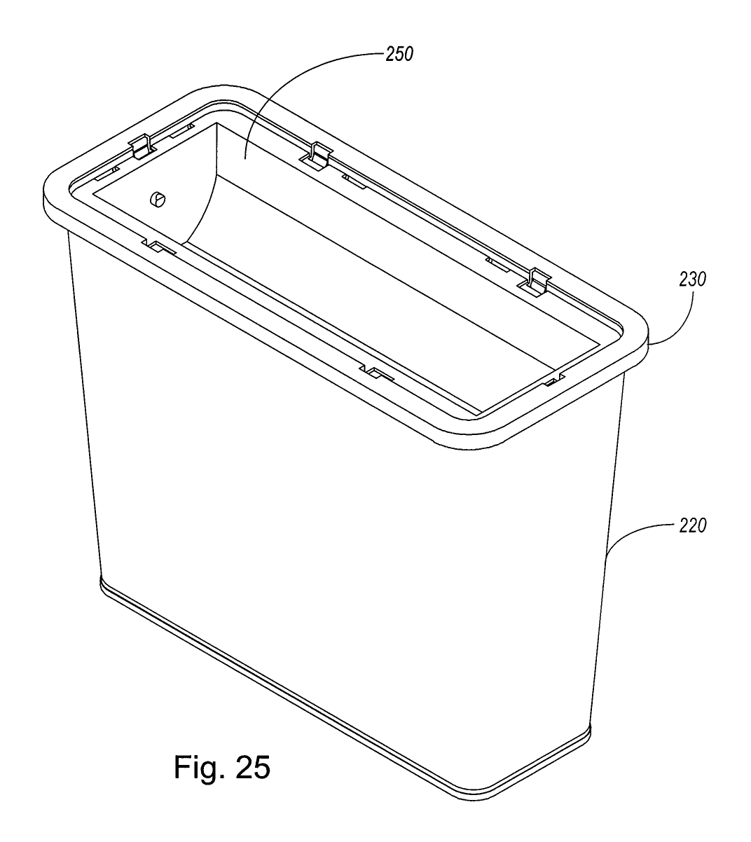

Chute 250 and spinner 260 may preferably be made of a transparent or partially transparent plastic in order to provide visual access to its contents, so, for example, persons using canister 220 can see when the canister is getting full. When used with a heat treatment process, such as those described above, chute 250 and spinner 260 are preferably made of a plastic material that will melt during an applied dry heat cycle (e.g., polyethylene, polypropylene, polycarbonate) beginning at temperatures of about 325 to about 350 degrees Fahrenheit. As the plastic material melts, it encapsulates the waste material that has been deposited in canister 220. When installed in canister 220, as shown in FIG. 26, chute 250 and spinner 260 limit access to the interior of the canister. FIG. 25 shows chute 250, without spinner 260, installed in canister 220.

Chute 250 has a tapered body 252 that is open at the top to receive waste and open at the bottom to allow waste to move through and into canister 220. Posts 254 are located on the interior surface of the short ends of the chute. Spinner 260 is mounted on posts 254 such that spinner 260 is rotatable about their center axis. Chute 250 includes a flange 256 at its top end. Flange 256 contacts inner portion 234 of rim 230 when chute 250 is installed within rim 230 at the top end of canister 220. Flange 256 includes a series of notches 257 configured so that chute 250 does not interfere with locations about rim 230 where rim 230 is designed to engage with plug 240. This is useful, for example, during heat processing of a sealed canister because chute 250 is preferably made of a material that melts away while rim 230 and plug 240 are designed to withstand the heat processing and remain intact. Tabs 258 are present on each of the long sides of chute 250 for engaging chute 250 with rim 230. As shown in FIGS. 21 and 22, tabs 258 extend down from flange 256 at two notches 257 on each long side of chute 250. Tabs 258 engage rim 230 from underneath inner portion 234 of rim 230, thereby retaining chute 250 within rim 230.

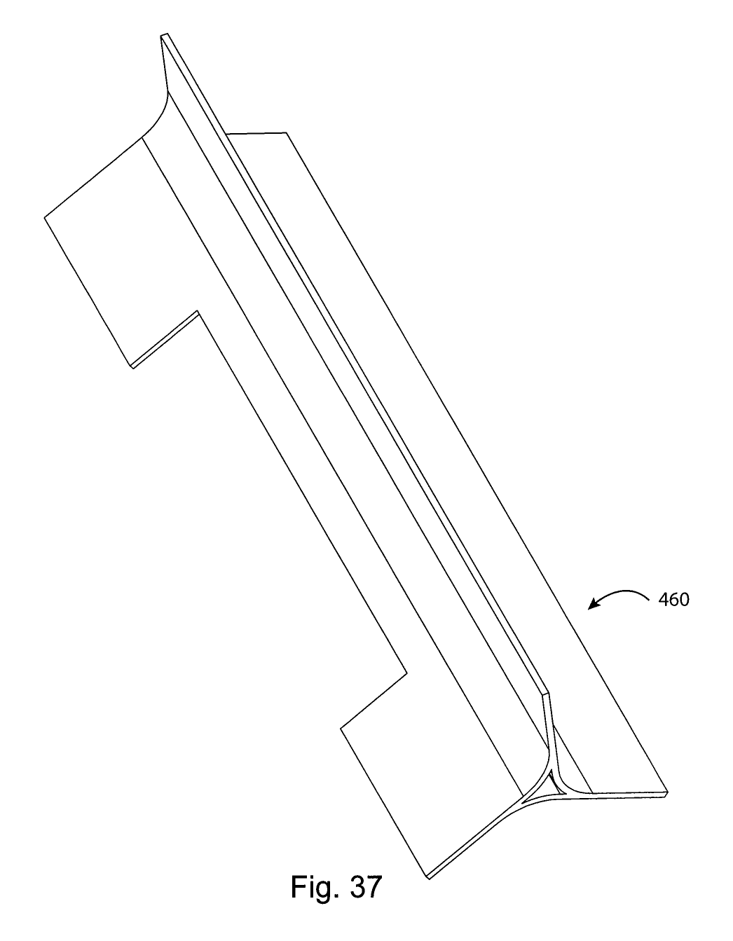

Spinner 260, shown best in FIGS. 21-23, includes holes 262 in each end that receive posts 254 of chute 250 for movably mounting spinner 260 within chute 250. Spinner 260 includes three fins 264 that extend out generally radially from the center of spinner 260. Waste is placed between two fins. The cross section of spinner 260 is designed to ensure unassisted rotation when waste is placed into spinner 260 by offsetting the low point of each fin 264 from the central axis of rotation of spinner 260. Thus, in use, when sharps material is placed onto a fin 264, the weight of the sharps material will cause the spinner to rotate. The rotation of spinner 260 causes the sharps material to drop through the bottom of chute 250 and into the portion of canister 220 that is beneath spinner 260, where the sharps material is then inaccessible from outside canister 220. Spinner 260 is dimensioned to fit within chute 250 such that a person's hand cannot easily reach around one of fins 264, through chute 250, and into the interior of canister 220. It should be understood that a suitable member of different shape or configuration may be used within the chute other than spinner 260, provided that such member can receive waste, deposit received waste into the portion of the canister beneath, and adequately limits access to the interior of the canister. One such embodiment 460 could resemble spinner 260, but with some or all of one of the blades removed to allow receipt of irregularly shaped waste types as shown in FIG. 37.

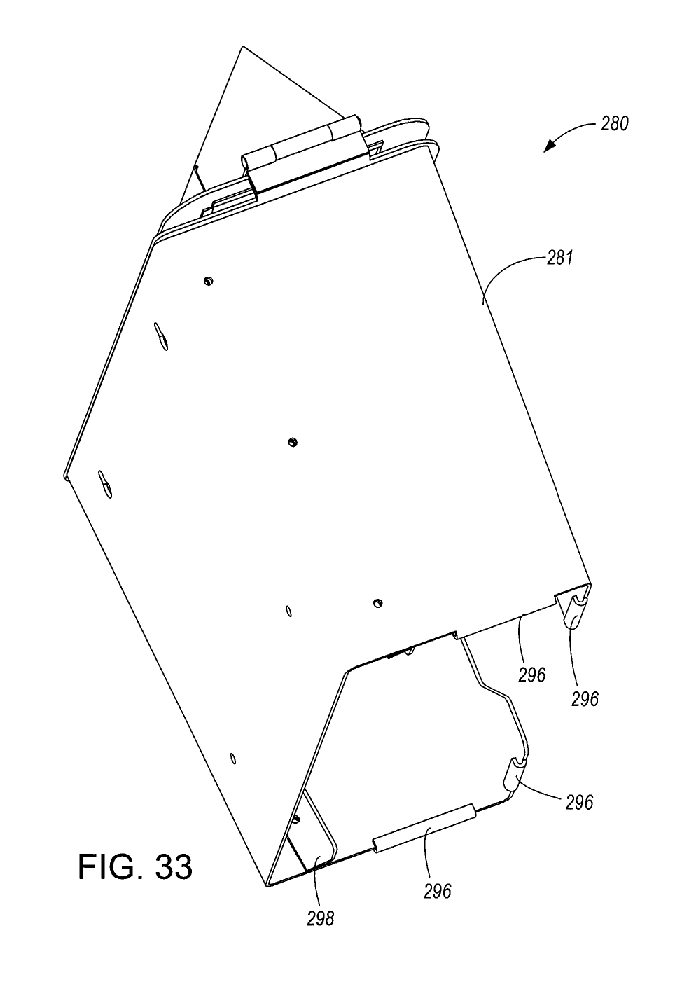

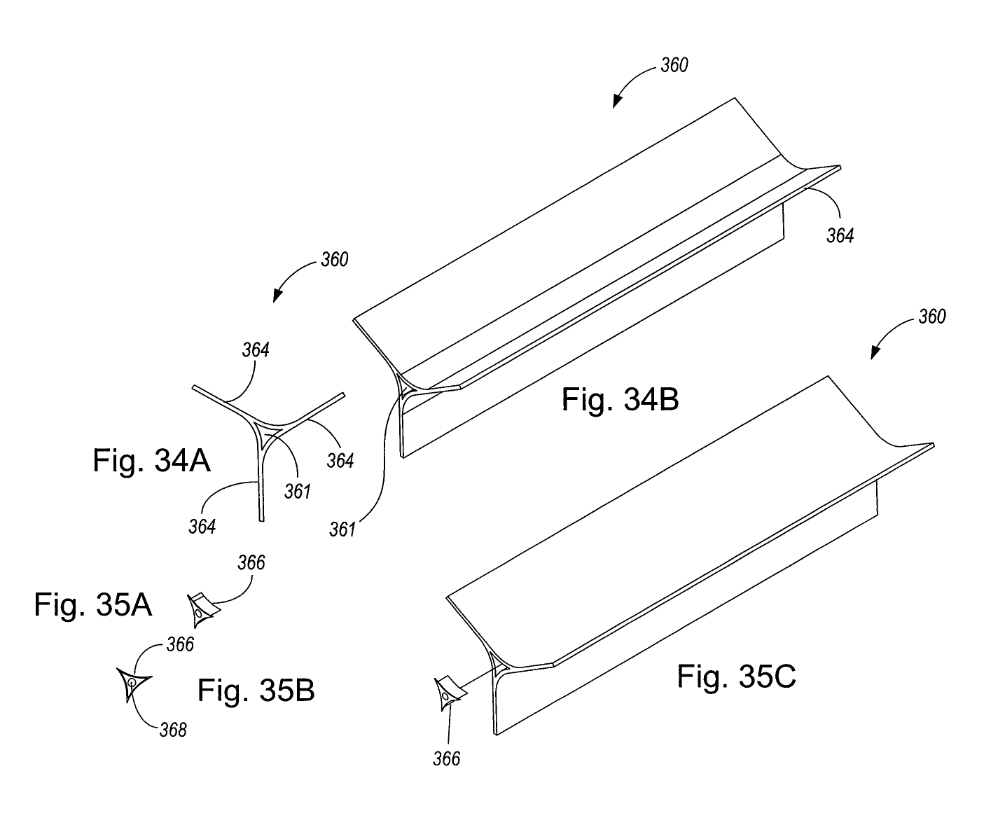

FIGS. 34A, 34B, and 35C show an alternative embodiment of a spinner 360 that may be mounted within a chute according to this invention. FIGS. 35A-35B show an insert 366 that fits within each aperture 361 in spinner 360. Spinner 360 had three fins 364 and works in the same general manner as spinner 260 described above. Spinner 360 has a hollow core that runs through the central axis of spinner 360, resulting in apertures 361 in each end of spinner 360. In a preferred embodiment, spinner 360 has a uniform wall thickness, which may help prevent warping of this component during manufacture. To mount spinner 360 within a chute, such as chute 250, an insert 366 is placed within each aperture 361. Inserts 366 each have a hole 368 that receives a post 254 of chute 250 so that spinner 360 is mounted within chute 250 and able to spin freely.

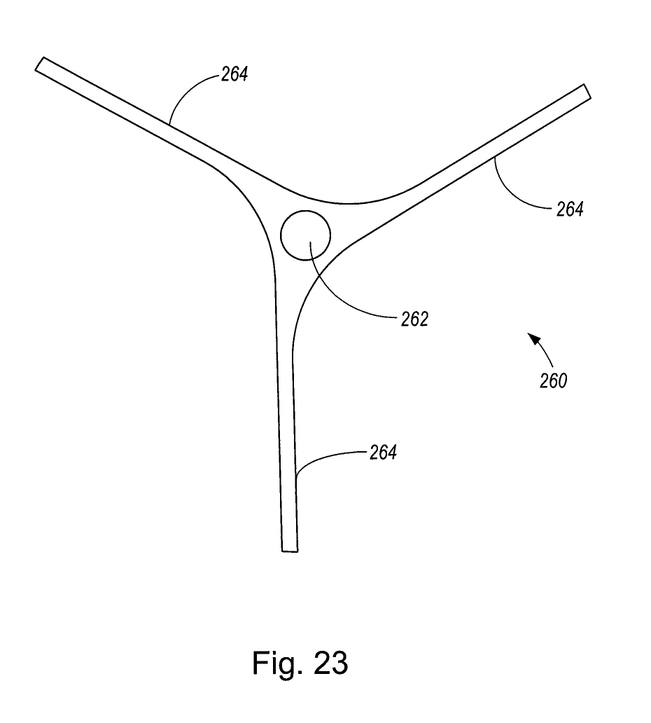

FIGS. 24A and 24B are perspective and end views, respectively, of rim 230, plug 240, chute 250, and spinner 260 assembled. In use, this assembly would be atop a canister, such as canister 220, when the canister is full and ready to be heat processed or otherwise disposed of.

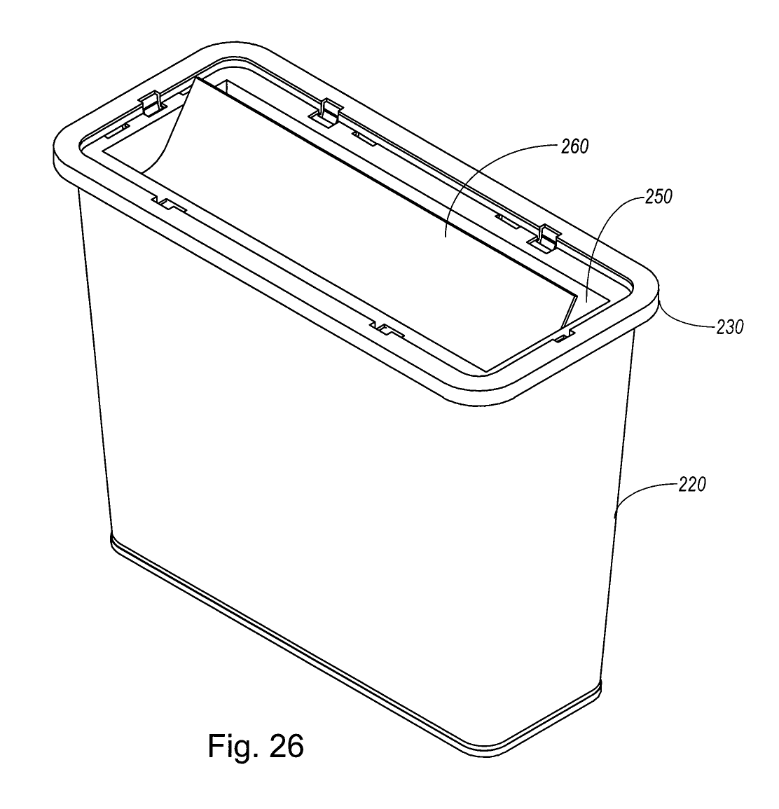

In another embodiment, shown in FIG. 27, a chute 270 is used with rim 230 within canister 220, and plug 240 is used to seal the canister when it is full or as otherwise desired. Chute 270 includes an opening 271 for receiving sharps material or other waste, and an opening 273 for allowing such waste to be collected in the interior of a canister with which it is being used. Chute 270 includes a flange 276 at its top end, flange 276 contacting inner portion 234 of rim 230 when chute 270 is installed within rim 230 at the top end of canister 220. Flange 276 includes a series of notches 277 configured so that chute 270 does not interfere with locations about rim 230 where rim 230 is designed to engage with plug 240. Tabs 278 are present on each of the long sides of chute 270 for engaging chute 270 with rim 230 from underneath inner portion 234 of rim 230, thereby retaining chute 250 within rim 230. An optional removable plastic funnel (not shown) may be used to assist in the loading of sharps material. Chute 270 may preferably be made of a transparent plastic in order to provide visual access to its contents and/or of a plastic material that will melt during an applied heat cycle so that the melted plastic encapsulates the waste material that has been deposited in canister 220.

Certain embodiments for use in collection of sharps material may include other components configured to provide for safe loading of sharps material. In particular, an outer container that may be mounted to a wall or other surface and is capable of being securely locked may be used to house a container for holding medical waste and other components in order to safely and effectively collect medical waste. The use of an outer container may be particularly desirable in public facilities or other settings in which many persons have access to the containers that hold the medical waste.

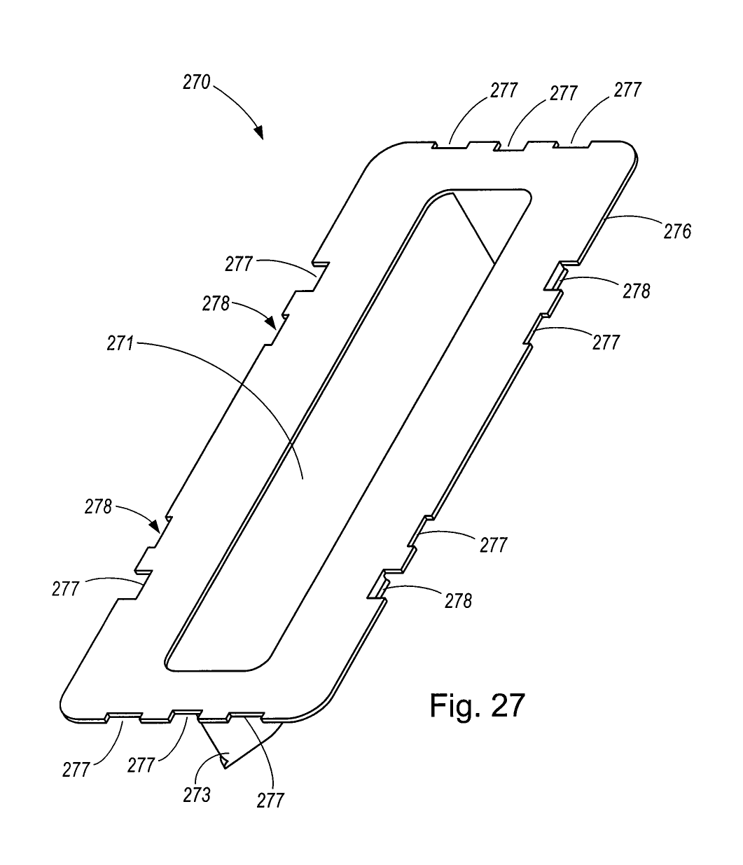

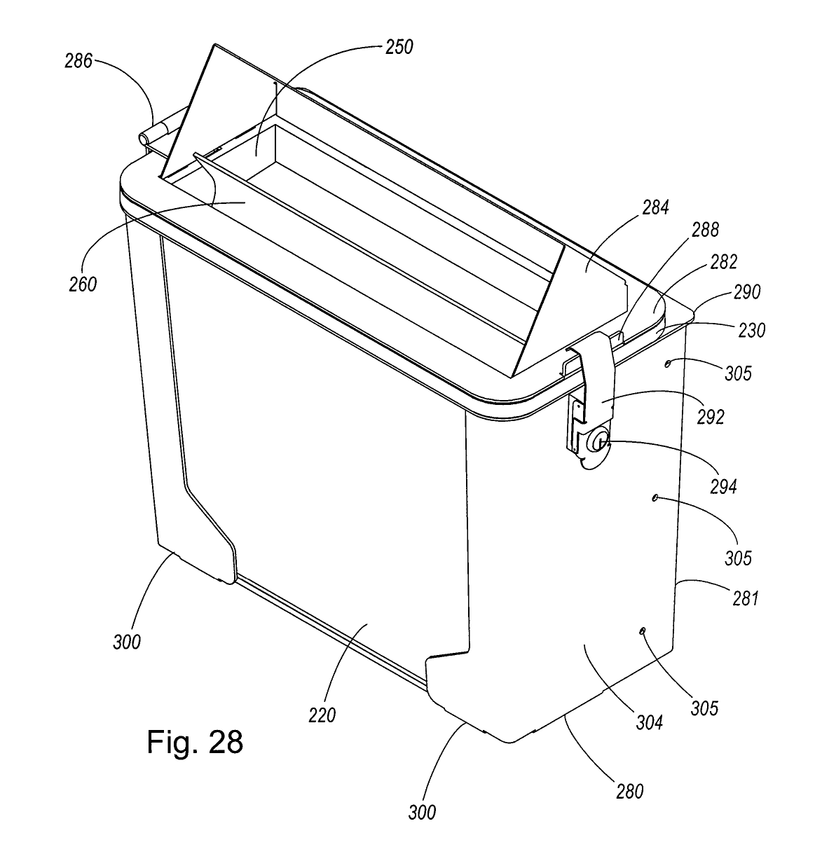

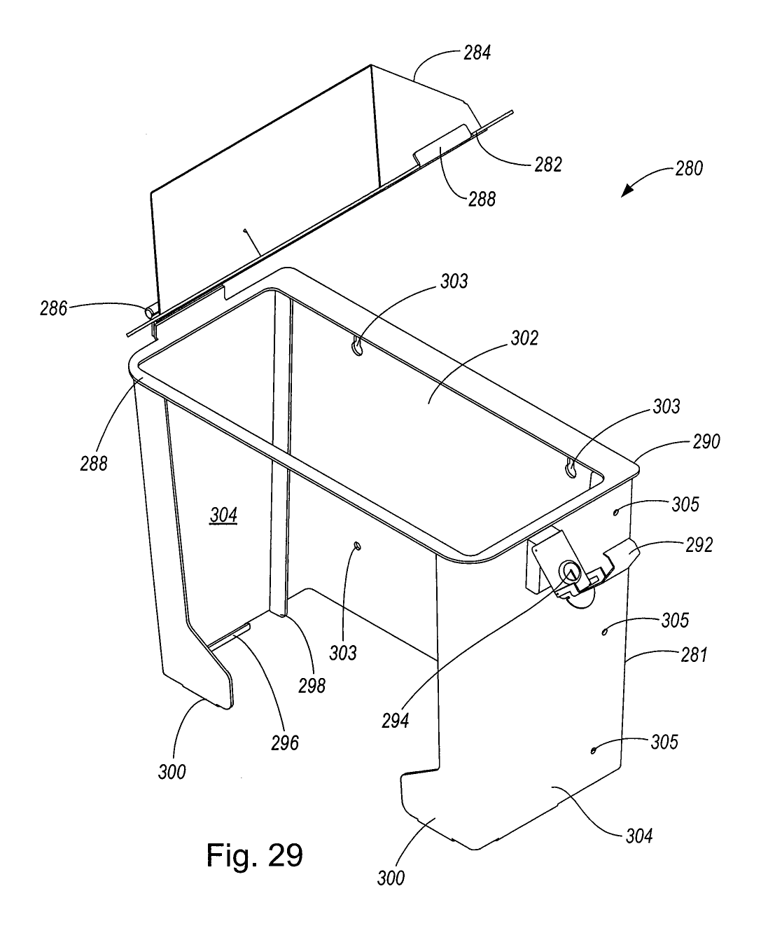

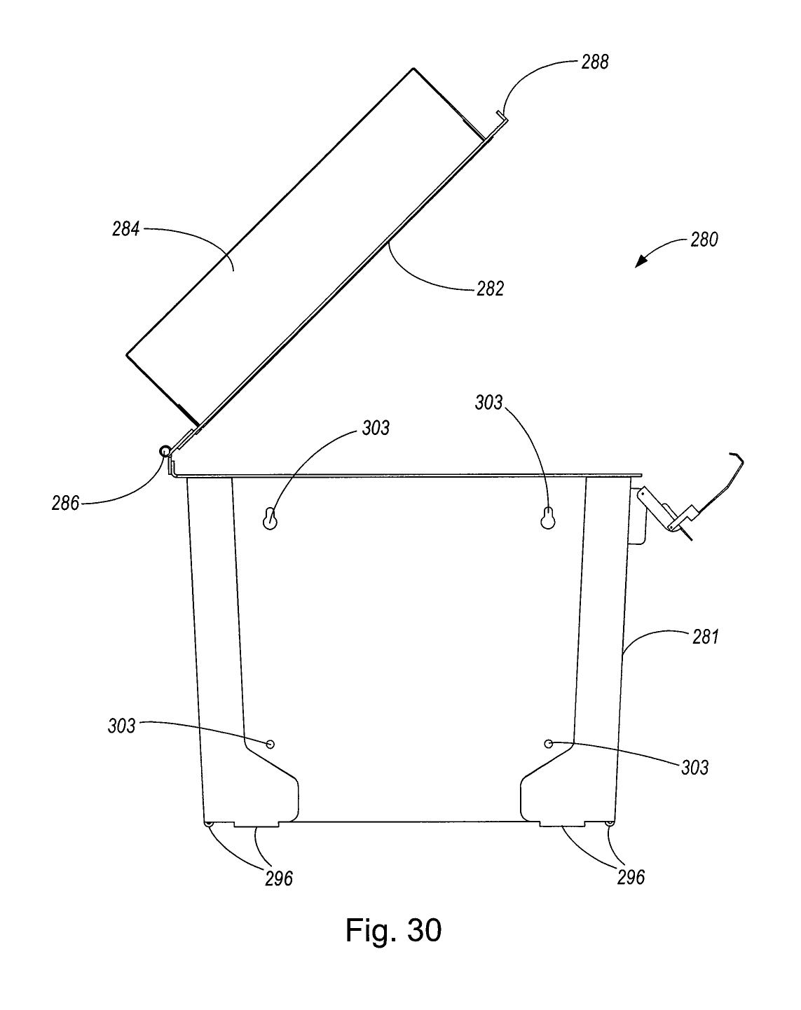

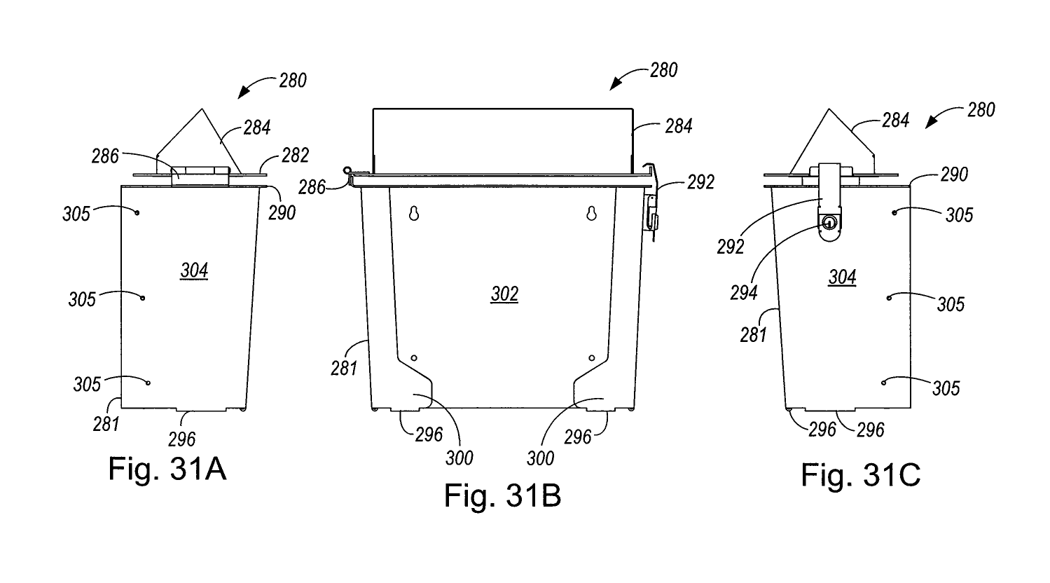

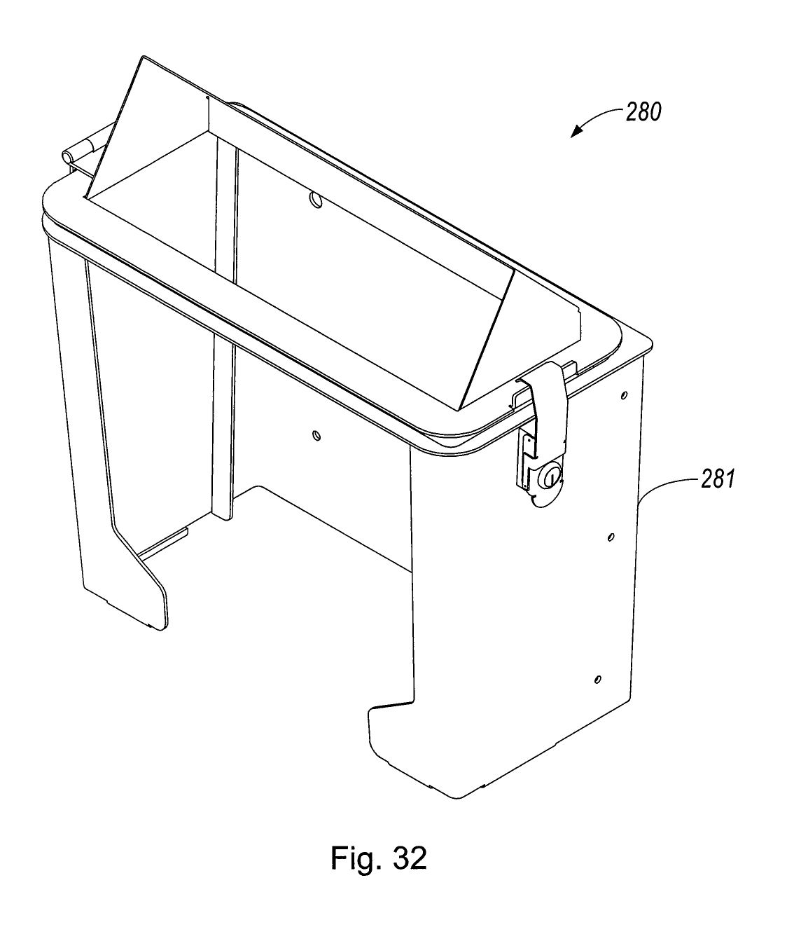

Referring now to FIGS. 28-33, an outer container 280 is shown. Outer container 280 is generally shaped to receive canister 220, but it should be understood that an outer container according to this invention may vary in shape provided that it is capable of receiving a canister for holding medical waste. As shown in FIG. 28, canister 220, with rim 230, chute 250, and spinner 260 assembled therewith, is placed within outer container 280. FIGS. 29-33 show outer container 280 without canister 220 and other associated components placed in outer container 280.

Outer container 280 includes a body 281 and a top 282. Top 282 is coupled to a flange 290 of body 281 by a hinge 286. This allows top 282 to open and close so that body 281 may receive a container for holding medical waste, such as canister 220, and the container for holding medical waste may be easily removed from outer container 280 when full or otherwise desired. In use in this embodiment, rim 230 sits upon flange 290 of body 281 of outer container 280, and top 282 is closed such that a bottom surface of top 282 contacts the top surface of rim 230. A latch 292 is mounted to body 281 and is moved so that it contacts a catch 288 on top 282 of outer container 280. A keylock 294 allows the movement of latch 292 to be locked, thereby securing canister 220, rim 230, chute 250, and spinner 260 within outer container 280. Top 282 includes a hood 284 through which medical waste is deposited. The medical waste contacts spinner 260, which rotates and dumps the waste through chute 250 and into canister 220, as described above.

In this particular embodiment, holes 303 are provided in a back side 302 of body 281 for mounting outer container 280 to a wall or other surface. Sides 304 of body 281 are each shown to have holes 305, which are used to rivet a bracket 298 onto the interior surface of each side 304. Brackets 298 provide support for canister 220 when canister 220 is placed within outer container 280. Extending from the bottom of sides 304 and front portions 300 of body 281 are support members 296. In this embodiment, support members 296 are generally u-shaped or j-shaped and receive the bottom edges of a canister 220 placed within the outer container 280.

In another embodiment of a canister shown in FIG. 36, canister 420 is made from a thermoplastic material that melts and encapsulates the medical waste upon the application of dry heat, in contrast to canister 220 that is designed to withstand the dry heat cycle. Otherwise, canister 420 has a similar or the same shape as canister 220. The rim may also be integrated into the rim of the canister rather than being a separate component snapping over the edge of the canister. Sleeve 410 may be constructed of aluminum and the interior coated with a non-stick material and is generally of a shape complementary to that of the interior of chamber 60. However, this embodiment is not limited to such materials or shapes, as noted above. Canister 420 is inserted into sleeve 410, and the combination is then inserted into chamber 60 where the dry heat is applied. Upon cooling down, sleeve 410 is removed from chamber 60, and the processed contents, formally canister 420 and waste within, may be discharged, and sleeve 410 reused in heat treatments of other canisters. When canister 420 is used, structures for safe disposal and collection of sharps material and other waste are used within the top of canister 420, similar to such use with canister 220. However, a plug, such as plug 240, is not used because all of the canister, rim, chute, and other components are made a plastic material that melts and encapsulates the waste, and that can then be removed from sleeve 410 and disposed.

The foregoing description of the exemplary embodiments of the invention has been presented only for the purposes of illustration and description and is not intended to be exhaustive or to limit the invention to the precise forms disclosed. Many modifications and variations are possible in light of the above teaching. The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope.

What is claimed is:

1. A system for thermal processing of infectious and medical waste, the system comprising:
a body having a chamber to receive a container of medical waste;
at least one plate heater coupled to an exterior surface of the chamber for providing heat to the container; and
a plurality of fins formed on an exterior surface of at least one side of the chamber for cooling the chamber.

2. The system of claim 1, further comprising a filter within the body and having an inlet coupled to the chamber.

3. The system of claim 1, wherein the at least one plate heater comprises two plate heaters, each plate heater coupled to an exterior surface of opposite sides of the chamber, and the system further comprises:
a plurality of fins on the exterior surfaces of the opposite sides of the chamber adjacent to where the two plate heaters are coupled and in an exterior surface on a bottom of the chamber.

4. The system of claim 1, further comprising a container comprising a canister for receiving medical waste.

5. The system of claim 4, wherein the chamber is shaped such that a plurality of interior surfaces of the chamber contact a plurality of exterior surfaces of the container when the container is received within the chamber.

6. The system of claim 4, wherein the container is dimensionally stable and heat resistant.

7. The system of claim 4, wherein the container comprises a thermoplastic material that melts to encapsulate sharps waste when heat is applied.

8. The system of claim 1, further comprising:
a container comprising a canister for receiving medical waste and made of a thermoplastic material; and
a sleeve shaped such that it receives the container and comes in contact with a plurality of the exterior surfaces of the container;
wherein the chamber is shaped such that a plurality of interior surfaces of the chamber contact a plurality of exterior surfaces of the sleeve when the sleeve is received within the chamber.

9. The system of claim 1, further comprising:
an exhaust tube extending from the chamber for exhaust resulting from heating the waste;
a second tube coupled to the exhaust tube, the second tube including a coiled portion that ends at a T-valve, wherein the exhaust from the chamber is separated into gas and condensed water in the second tube;
a third tube coupled to one outlet of the T-valve, the third tube for passing exhaust gas to a filter; and
a fourth tube coupled to another outlet of the T-valve, the fourth tube for passing liquid to a collection receptacle.

10. The system of claim 1, further comprising a processor coupled to an external link capable of contact with a remote database and memory for storing data captured during a treatment cycle, wherein the memory comprises three data storage buffers, one for short term data, one for intermediate term data, and one for long term data.

11. A method for heat-processing infectious and medical waste, comprising:
heating a container of waste in a chamber to render the waste biologically safe using at least one plate heater coupled to an exterior surface of a side of the chamber; and
cooling the chamber using a plurality of fins positioned on an exterior surface of at least one side of the chamber and a plurality of fins positioned an exterior surface of a bottom of the chamber.

12. The method of claim 11, further comprising providing the chamber to receive the container of waste, the chamber being shaped complementary to the shape of the container so that a plurality of exterior surfaces of the container contact a plurality of interior surfaces of the chamber when the container is received within the chamber.

13. The method of claim 11, further comprising providing the chamber to receive the container of waste and providing a reusable sleeve within the chamber to receive the container of waste, wherein the sleeve is shaped complementary to the interior of the chamber and the exterior of the container.

14. The method of claim 11, wherein heating the container of waste further comprises heating the chamber to a temperature of about 350 to about 400 degrees Fahrenheit.

15. The method of claim 11, further comprising:
separating exhaust in the chamber resulting from heating the waste into gas and condensed water; and
directing the gas through a tube to a filter and directing the condensed water into a collection receptacle.

16. The method of claim 11, further comprising:
storing data related to a treatment cycle during which a container of waste is heated and cooled; and
transmitting at least a portion of the stored data to a remote database.

17. An apparatus for collection of infectious and medical waste, the apparatus comprising:
a canister comprising a top end with an opening for receiving waste;
a rim configured to fit over an edge of and engage the top end of the canister; and
a plug configured to engage the rim such that the plug seals the opening of the canister.

18. The apparatus of claim 17, further comprising:
a chute configured to engage an inner portion of the rim, the chute extending into an interior of the canister when engaged with the rim atop the canister; and
a member movably mounted within the chute and configured to limit access to a portion of the interior of the canister beneath the member.

19. The apparatus of claim 17, wherein:
the canister further comprises a plurality of slots spaced around a periphery of the canister; and
the rim further comprises a plurality of projections that are received within the plurality of slots of the canister when the rim is engaged with the canister.

20. The apparatus of claim 19, wherein the rim further comprises (a) a plurality of slots spaced around a periphery of an inner portion of the rim, and (b) a plurality of apertures just above the plurality of projections; and the plug further comprises (a) a plurality of hooks that are received within the plurality of slots of the rim when the plug is engaged with the rim and (b) a plurality of tabs that are received within the apertures of the rim when the plug is engaged with the rim.

21. The apparatus of claim 18, wherein the chute further comprises a flange that sits upon an inner portion of the rim when the chute and the rim are engaged and posts to which the member is mounted, the flange comprising a plurality of notches spaced around the flange and positioned to avoid interfering with engagement of the rim and the plug.

22. The apparatus of claim 18, wherein the member further comprises a spinner with a plurality of fins defining areas that receive the waste, each fin being configured to rotate and dump waste into the portion of the interior of the canister beneath the member when waste is placed into one of the areas.

23. A system for thermal processing of infectious and medical waste, the system comprising:
a body having a chamber;
a sleeve configured to be received within the chamber; and
a canister for receiving medical waste, wherein the canister is configured to fit within the sleeve and is made of a thermoplastic material that melts upon heating the chamber to a high temperature and then solidifies upon cooling to encapsulate any waste within the canister.

24. The system of claim 23, wherein the sleeve is dimensionally stable and heat resistant.

25. The system of claim 23, wherein the sleeve is configured such that a plurality of its exterior surfaces are in contact with a plurality of interior surfaces of the chamber.
